SEQUENCE LISTING (41 sequences)

SEQ ID NO: 1 - protein, 41 aa, Vipera lebetina obtusa
CTTGPCCRQCKLKPAGTTCWKTSLTSHYCTGKSCDCPLYPG

SEQ ID NO: 2 - protein, 41 aa, Vipera palestinae
CTTGPCCRQCKLKPAGTTCWKTSRTSHYCTGKSCDCPVYQG

SEQ ID NO: 3 - protein, 20 aa, Artificial Sequence
  N-terminal KTS-disintegrin fragment
CTTGPCCRQCKLKPAGTTCW

SEQ ID NO: 4 - protein, 18 aa, Artificial Sequence
  C-terminal KTS-disintegrin fragment
XTSHYCTGKSCDCPXYXG

SEQ ID NO: 5 - protein, 11 aa, Artificial Sequence
  obtustatin fragment
CWKTSLTSHYC

SEQ ID NO: 6 - protein, 5 aa, Artificial Sequence
  obtustatin fragment
TSLTS

SEQ ID NO: 7 - protein, 10 aa, Artificial Sequence
  obtustatin fragment
CKLKPAGTTC

SEQ ID NO: 8 - protein, 19 aa, Artificial Sequence
  N-terminal KTS-disintegrin fragment
TTGPCCRQCKLKPAGTTCW

SEQ ID NO: 9 - protein, 18 aa, Artificial Sequence
  N-terminal KTS-disintegrin fragment
TGPCCRQCKLKPAGTTCW

SEQ ID NO: 10 - protein, 17 aa, Artificial Sequence
  N-terminal KTS-disintegrin fragment
GPCCRQCKLKPAGTTCW

SEQ ID NO: 11 - protein, 16 aa, Artificial Sequence
  N-terminal KTS-disintegrin fragment
PCCRQCKLKPAGTTCW

SEQ ID NO: 12 - protein, 15 aa, Artificial Sequence
  N-terminal KTS-disintegrin fragment
CCRQCKLKPAGTTCW

SEQ ID NO: 13 - protein, 14 aa, Artificial Sequence
  N-terminal KTS-disintegrin fragment
CRQCKLKPAGTTCW

SEQ ID NO: 14 - protein, 13 aa, Artificial Sequence
  N-terminal KTS-disintegrin fragment
RQCKLKPAGTTCW

SEQ ID NO: 15 - protein, 12 aa, Artificial Sequence
  N-terminal KTS-disintegrin fragment
QCKLKPAGTTCW

SEQ ID NO: 16 - protein, 11 aa, Artificial Sequence
  N-terminal KTS-disintegrin fragment
CKLKPAGTTCW

SEQ ID NO: 17 - protein, 10 aa, Artificial Sequence
  N-terminal KTS-disintegrin fragment
KLKPAGTTCW

SEQ ID NO: 18 - protein, 9 aa, Artificial Sequence
  N-terminal KTS-disintegrin fragment
LKPAGTTCW

SEQ ID NO: 19 - protein, 8 aa, Artificial Sequence
  N-terminal KTS-disintegrin fragment
KPAGTTCW

SEQ ID NO: 20 - protein, 7 aa, Artificial Sequence
  N-terminal KTS-disintegrin fragment
PAGTTCW

SEQ ID NO: 21 - protein, 6 aa, Artificial Sequence
  N-terminal KTS-disintegrin fragment
AGTTCW

SEQ ID NO: 22 - protein, 5 aa, Artificial Sequence
  N-terminal KTS-disintegrin fragment
GTTCW

SEQ ID NO: 23 - protein, 4 aa, Artificial Sequence
  N-terminal KTS-disintegrin fragment
TTCW

SEQ ID NO: 24 - protein, 17 aa, Artificial Sequence
  C-terminal KTS-disintegrin fragment
XTSHYCTGKSCDCPXYX

SEQ ID NO: 25 - protein, 16 aa, Artificial Sequence
  C-terminal KTS-disintegrin fragment
XTSHYCTGKSCDCPXY

SEQ ID NO: 26 - protein, 15 aa, Artificial Sequence
  C-terminal KTS-disintegrin fragment
XTSHYCTGKSCDCPX

SEQ ID NO: 27 - protein, 14 aa, Artificial Sequence
  C-terminal KTS-disintegrin fragment
XTSHYCTGKSCDCP

SEQ ID NO: 28 - protein, 13 aa, Artificial Sequence
  C-terminal KTS-disintegrin fragment
XTSHYCTGKSCDC

SEQ ID NO: 29 - protein, 12 aa, Artificial Sequence
  C-terminal KTS-disintegrin fragment
XTSHYCTGKSCD

SEQ ID NO: 30 - protein, 11 aa, Artificial Sequence
  C-terminal KTS-disintegrin fragment
XTSHYCTGKSC

SEQ ID NO: 31 - protein, 10 aa, Artificial Sequence
  C-terminal KTS-disintegrin fragment
XTSHYCTGKS

SEQ ID NO: 32 - protein, 9 aa, Artificial Sequence
  C-terminal KTS-disintegrin fragment
XTSHYCTGK

SEQ ID NO: 33 - protein, 8 aa, Artificial Sequence
  C-terminal KTS-disintegrin fragment
XTSHYCTG

SEQ ID NO: 34 - protein, 7 aa, Artificial Sequence
  C-terminal KTS-disintegrin fragment
XTSHYCT

SEQ ID NO: 35 - protein, 6 aa, Artificial Sequence
  C-terminal KTS-disintegrin fragment
XTSHYC

SEQ ID NO: 36 - protein, 5 aa, Artificial Sequence
  C-terminal KTS-disintegrin fragment
XTSHY

SEQ ID NO: 37 - protein, 4 aa, Artificial Sequence
  C-terminal KTS-disintegrin fragment
XTSH

SEQ ID NO: 38 - protein, 20 aa, Artificial Sequence
  KTS-disintegrin fragment M10
KPAGTTCWKTSLTSHYCTGK

SEQ ID NO: 39 - protein, 10 aa, Artificial Sequence
  KTS-disintegrin fragment S14
CWKTSLTSHY

SEQ ID NO: 40 - protein, 33 aa, Artificial Sequence
  KTS-disintegrin fragment F4
CCRQCKLKPAGTTCWKTSRTSHYCTGKSCDCPV

SEQ ID NO: 41 - protein, 37 aa, Artificial Sequence
  KTS-disintegrin cysteine skeleton
CXXXXCCXXCXXXXXXXXCXKTSXXXXXCXXXXCXCX

(19) United States
(12) Patent Application Publication
(10) Pub. No.: US 2003/0186334 A1
(43) Pub. Date: Oct. 2, 2003

(54) KTS-disintegrins

(75) Inventor: Cezary Marcinkiewicz, King of Prussia, PA (US)

Correspondence Address: DRINKER BIDDLE & REATH, ONE LOGAN SQUARE, 18TH AND CHERRY STREETS, PHILADELPHIA, PA, 19103-6996, US

(73) Assignee: Temple University - Of the Commonwealth System of Higher Education, Philadelphia, PA (US)

(21) Appl. No.: 10/386,055

(22) Filed: Mar. 11, 2003

Related U.S. Application Data
(63) Continuation-in-part of application No. PCT/US01/28522, filed on Sep. 12, 2001.

(51) Int. Cl. G01N 33/567

(52) U.S. Cl. 435/7.2

(57) ABSTRACT

A novel class of non-RGD disintegrins is described. These disintegrins have the core sequence KTS, and selectively inhibit the binding of α1β1 integrin to its adhesive ligands. Two KTS-disintegrins, obtustatin and viperisrastatin, are also described. KTS-disintegrins can be used to treat diseases and modulate biological conditions associated with α1β1 integrin.

### CROSS REFERENCE TO RELATED APPLICATIONS

This application is a continuation-in-part of copending International patent application PCT/US01/28522, filed Sep. 12, 2001 and which published in English under PCT Article 21(2) on Mar. 21, 2002, which claims the benefit of U.S. Provisional Application Serial No. 60/231,591 filed Sep. 11, 2000.

### REFERENCE TO GOVERNMENT GRANT

The invention described herein was supported in part by the National Institutes of Health, under grant no. RSO1 HL 60921-01. The U.S. government has certain rights in this invention.

### FIELD OF THE INVENTION

This invention relates to methods and compositions for modulating cell adhesion, and for inhibiting the interaction between integrins and their ligands. In particular, the invention relates to small KTS-disintegrins and their use in inhibiting interaction of α1β1 integrin with its ligand.

### BACKGROUND OF THE INVENTION

Integrins are a family of cell surface proteins that mediate adhesion between cells (cell-cell adhesion) and between cells and the extracellular matrix (cell-ECM adhesion). The known integrins are heterodimeric proteins composed of noncovalently bound α and β subunits. In humans there are at least 15 different α and eight different β subunits, which can combine to form unique integrins with diverse biological activities and ligand specificities.

The integrins mediate cell-cell and cell-ECM adhesion by binding to adhesive ligands carried by cells or found in the ECM. Examples of adhesive ligands include fibrinogen, fibronectin, collagen I, collagen IV, and vascular cell adhesion molecule-1 (VCAM-1). The integrins play an important role in many diverse biological processes, including platelet-aggregation, tissue repair, angiogenesis, bone destruction, tumor invasion, inflammation, restenosis of the arteries following surgery or angioplasty, and immune reactions. Furthermore, integrin binding activity has been implicated in a number of disease states, including coronary thrombosis, atherosclerotic diseases, vascular disease, heart disease, diabetes, multiple sclerosis, rheumatoid arthritis, ulcerative colitis, arteriosclerosis, asthma, and autoimmune disorders. Consequently, integrins are important targets for therapeutic intervention in human disease and for regulation of normal biological processes.

The majority of integrins identified to date are “RGD-dependent integrins,” which bind to a three amino acid RGD (arginine-glycine-aspartic acid) sequence found in their respective adhesive ligands. The αIIbβ, αvβ3 and α5β1 integrins are all examples of RGD-dependent integrins. Integrin αIIbβ3 binds fibrinogen on the surface of platelets and mediates platelet-aggregation. Integrin αvβ3 is predominantly expressed on endothelial cells (where it is involved in angiogenesis) and osteoclasts (where it participates in bone destruction). Integrin α5β1 is expressed by a variety of cell types and is involved in cell adhesion to the extracellular matrix as well as in the formation of tissues and organs during embryonic development.

Some integrins bind to sequences other than RGD. Such integrins are classified as “RGD-independent integrins.” An important RGD-independent integrin is the α1β1 integrin. The adhesive ligand of α1β1 integrin is collagen IV, and in some cases collagen I. To date, the sequence on collagen I or IV to which the α1β1 integrin binds has not been identified.

The α1β1 integrin is expressed by a variety of cell types and is involved in angiogenesis, vascularization of tissues, and lymphocyte migration. This integrin is also involved in the formation of basement membranes and in the interaction of cells with these membranes. Activation and up-regulation of α1β1 integrin in lymphocytes or macrophages is also believed to play a significant role in the progression of many disease states and biological processes, including insulin dependent diabetes mellitus, multiple sclerosis, rheumatoid arthritis, ulcerative colitis, arteriosclerosis, asthma, allergy, organ rejection, restenosis of arteries after surgery or angioplasty, and angiogenesis.

Angiogenesis is the process in which new blood vessels grow into an area which lacks a sufficient blood supply. Angiogenesis commences with the erosion of the basement membrane surrounding endothelial cells and pericytes of existing capillary blood vessels. Erosion of the basement membrane is triggered by enzymes released by endothelial cells and leukocytes. The endothelial cells then migrate through the eroded basement membrane when activated by angiogenic stimulants. The migrating cells form a “sprout” off the parent blood vessel. The migrating endothelial cells proliferate, and the sprouts merge to form capillary loops, thus forming a new blood vessel.

Angiogenesis can occur under certain normal conditions in mammals such as in wound healing, in fetal and embryonic development, and in the formation of the corpus luteum, endometrium and placenta. Angiogenesis also occurs in certain disease states such as in tumor formation and expansion, or in the retina of patients with certain ocular disorders. Angiogenesis can also occur in a rheumatoid joint, hastening joint destruction by allowing an influx of leukocytes with subsequent release of inflammatory mediators. The role of angiogenesis in tumor growth was extensively reviewed by O'Reilly and Folkman in U.S. Pat. No.5,639,725, the entire disclosure of which is incorporated herein by reference. It is now generally accepted that the growth of tumors is critically dependent upon angiogenesis.

The disintegrins are a family of low molecular weight, soluble cysteine-rich peptides that interfere with the binding of integrins to their adhesive ligands. Disintegrins carry sequences identical or analogous to the binding sites in the adhesive ligands of integrins, and bind integrins with an affinity comparable to that of monoclonal antibodies. Many disintegrins have been isolated from the venom of various snakes, as well as other sources, and several disintegrin subfamilies have been identified. These disintegrin subfamilies differ from each other on the basis of peptide chain length, the number of conserved cysteines, dimerization state and type of integrin binding site (reviewed in McLane et al., P.S.E.B.M. 219:109-119 (1998)).

Another class of molecules called “cellular disintegrins” has been identified in mammalian cells. The cellular disintegrins appear to be structurally and functionally distinct from the small soluble disintegrins. For example, the cellular disintegrins comprise multiple domains, and are generally much larger than the soluble disintegrins, and are typically localized to the cell membrane. One such cellular disintegrin, called MDC9 or ADAM-9, contains a metalloproteinase domain, a disintegrin domain, and an SH3 ligand domain. Wekamp G et al., J. Cell. Biol. 132: 717-726. A putative integrin binding site in the MDC9 disintegrin domain has been identified as the amino-acid sequence Thr-Ser-Glu-Cys (TSEC). An arginine (R), a glycine (G) and a lysine (K) are located immediately adjacent to the Thr of this putative binding sequence, in that order. The region of the MDC9 disintegrin domain containing the putative binding sequence thus has the sequence RGKTSEC.

Small soluble disintegrins containing the RGD sequence prevent the binding of RGD-dependent integrins to their adhesive ligands, presumably through a competition-type mechanism.. For example, the RGD motif is present in a biologically active fragment of the short disintegrin echistatin. This fragment prevents the RGD-dependent αIIbβ3 integrin from associating with the RGD sequence in its adhesive ligand fibrinogen.

Because disintegrins interfere with the binding of integrins to their adhesive ligands, they can potentially be used in the prevention and treatment of diseases involving integrin binding. Additionally, disintegrins can be used to affect normal integrin-mediated biological processes. However, the vast majority of disintegrins described to date contain an RGD sequence, or a conservative substitution of one of the residues present in the RGD sequence (such as the KGD, or lysine-glycine-aspartic acid, sequence of the disintegrin barbourin). Thus, only diseases or biological processes involving RGD-dependent integrins can be affected with these disintegrins.

In contrast, the treatment of diseases or the modulation of biological processes that involve RGD-independent integrins has heretofore been difficult. As discussed above, many disease states and biological processes are associated with RGD-independent integrins such as the α1β1 integrin. Consequently, there is a need for potent and specific inhibitors of α1β1 integrin, which can be used in the treatment of disease or the modulation of biological processes involving the α1β1 integrin.

### SUMMARY OF THE INVENTION

Short disintegrins comprising the core sequence KTS, instead of RGD, are potent and specific inhibitors of α1β1 integrin. Two such disintegrins are obtustatin and viperisrastatin. The invention thus provides KTS-disintegrins comprising obtustatin (SEQ ID NO: 1) or viperisrastatin (SEQ ID NO: 2), as well as biologically active fragments, derivatives, homologs and analogs of these disintegrins.

The invention also provides KTS-disintegrins comprising the sequence X₁-Lys-Thr-Ser-X₂, wherein X₁ is from zero to twenty-five amino acids, and X₂ is from zero to twenty amino acids. In one embodiment, X₁ can comprise zero amino acids, or the segment Cys-Thr-Thr-Gly-Pro-Cys-Cys-Arg-Gln-Cys-Lys-Leu-Lys-Pro-Ala-Gly-Thr-Thr-Cys-Trp (SEQ ID NO: 3) or an amino-terminal truncation fragment thereof containing at least one amino acid. In this same embodiment, X₂ is zero amino acids, or comprises the segment X₄-Thr-Ser-His-Tyr-Cys-Thr-Gly-Lys-Ser-Cys-Asp-Cys-Pro-X₅-Tyr-X₆-Gly (SEQ ID NO: 4) or a carboxy-terminal truncation fragment thereof containing at least one amino acid. X₄ is Leu or Arg, X₅ is Leu or Val, and X₆ is Pro or Gln; provided, when X₄ is Leu then X₅ is Leu and X₆ is Pro; and when X₄ is Arg then X₅ is Val and X₆ is Gln.

The invention also provides KTS-disintegrins comprising the sequence Cys-Xaa-Xaa-Xaa-Xaa-Cys-Cys-Xaa-Xaa-Cys-Xaa-Xaa-Xaa-Xaa-Xaa-Xaa-Xaa-Xaa—Cys-Xaa-Lys-Thr-Ser-Xaa-Xaa-Xaa-Xaa-Xaa-Cys-Xaa-Xaa-Xaa-Xaa-Cys-Xaa-Cys-X₇ (SEQ ID NO: 41), wherein X₇ is zero or any 1, 2, 3, 4, or 5 amino acids, and each Xaa is independently any amino acid.

The invention also provides a pharmaceutical compositions comprising a KTS-disintegrin, or a biologically active fragment, derivative, homolog or analog thereof, and a pharmaceutically acceptable carrier.

The invention also provides a method of inhibiting the binding of α1β1 integrins to their adhesive ligands, comprising contacting a sample with an effective amount of a KTS-disintegrin, or a biologically active fragment, derivative, homolog or analog thereof, so that the binding of α1β1 integrin to its adhesive ligands is inhibited.

The invention also provides a method of treating diseases or biological conditions associated with the binding of α1β1 integrins to their adhesive ligands, comprising administering to a subject an effective amount of a KTS-disintegrin or a biologically active fragment, derivative, homolog or analog thereof, sufficient to inhibit the binding of α1β1 integrin with its ligands.

The invention also provides a method of detecting α1β1 integrin in a sample, comprising contacting the sample with a labeled KTS-disintegrin for a time sufficient to allow binding of the labeled KTS-disintegrin to any α1β1 integrin present in the sample. Unbound labeled KTS-disintegrin is then removed, and the labeled a KTS-disintegrin which is bound to the α1β1 integrin in the sample is detected. The method can also be practiced with a labeled biologically active fragment, derivative, homolog or analog of a KTS-disintegrin.

The invention also provides a method of isolating α1β1 integrin from a sample, comprising contacting the sample with KTS-disintegrin which has been modified with a selectable label for a time sufficient for the modified KTS-disintegrin to bind to any α1β1 integrin present in the sample. The α1β1 integrin is then isolated by separating the modified KTS-disintegrin from the sample via the selectable label. The method can also be practiced with a biologically active fragment, derivative, homolog or analog of a KTS-disintegrin which has been modified with a selectable label.

The invention also provides antibodies against the present KTS-disintegrins, or biologically active fragments, derivatives, homologs or analogs thereof.

### Amino Acid Abbreviations

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified. In the amino acid structure formulae, each residue is generally represented by a one-letter or three-letter designation, corresponding to the trivial name of the amino acid, in accordance with the following schedule:

| | | |
|---|---|---|
| A | Alanine | Ala |
| C | Cysteine | Cys |
| D | Aspartic Acid | Asp |
| E | Glutamic Acid | Glu |
| F | Phenylalanine | Phe |
| G | Glycine | Gly |
| H | Histidine | His |
| I | Isoleucine | Ile |
| K | Lysine | Lys |
| L | Leucine | Leu |
| M | Methionine | Met |
| N | Asparagine | Asn |
| P | Proline | Pro |
| Q | Glutamine | Gln |
| R | Arginine | Arg |
| S | Serine | Ser |
| T | Threonine | Thr |
| V | Valine | Val |
| W | Tryptophan | Trp |
| Y | Tyrosine | Tyr |

### Definitions

The following definitions, of terms used throughout the specification, are intended as an aid to understanding the scope and practice of the present invention.

By “α1β1 integrin” is meant any integrin comprising an α1 subunit and a β1 subunit.

By “integrin” is meant a family of heterodimeric cell surface proteins which mediate adhesion between cells as well as between cells and extracellular matrix proteins.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half life without adversely affecting their activity. Additionally, a disulfide linkage can be present or absent in the peptides of the invention.

Amino acids have the following general structure:

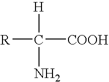

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

“Amino-terminal truncation fragment” with respect to an amino acid sequence means a fragment obtained from a parent sequence by removing one or more amino acids from the amino-terminus thereof.

“Analogs” of are small molecule compounds which exhibit one or more biological activities of a reference peptide.

The terms “antibodies” or “antibody,” as used herein, refer to intact immunoglobin molecules, as well as fragments of immunoglobulin molecules, such as Fab, Fab′, (Fab′)₂, Fv, and SCA fragments, which specifically bind to an epitope of those compounds of the invention which are peptides, fragments, derivatives or homologs.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

“Carboxy-terminal truncation fragment” with respect to an amino acid sequence means a fragment obtained from a parent sequence by removing one or more amino acids from the carboxy-terminus thereof.

“Biologically active” with respect to the compounds of the invention means the ability to inhibit α1β1 integrin-mediated cellular adhesion to collagen IV, for example as determined by the assay of Examples 4 and 5 below.

“Derivative” includes any purposefully generated peptide which in its entirety, or in part, has a substantially similar amino acid sequence to a reference peptide, and which retains at least one biological property of the reference peptide. Derivatives can be characterized by single or multiple amino acid substitutions, deletions, additions, or replacements. These derivatives can include (a) derivatives in which one or more amino acid residues are substituted with conservative or non-conservative amino acids, (b) derivatives in which one or more amino acids are added, (c) derivatives in which one or more of the amino acids includes a substituent group (including an N- or C-terminal protecting group), (d) derivatives in which the reference peptide or a portion thereof is fused to another peptide (e.g., serum albumin), (e) derivatives in which one or more nonstandard amino acid residues. (i.e., those other than the 20 standard L-amino acids commonly found in naturally occurring proteins) are incorporated or substituted into the reference peptide sequence, and (f) derivatives in which one or more nonamino acid linking groups are, incorporated into or replace a portion of the reference peptide.

“Fragment” refers to a portion of the reference peptide sequence comprising at least two amino acid residues of that sequence. Fragments can be generated by amino-terminal truncation, carboxy-terminal truncation, or both. Fragments can also be generated by chemical or enzymatic digestion.

“Homolog” includes any nonpurposely generated peptide which in its entirety, or in part, has a substantially similar amino acid sequence to a reference peptide and exhibits at least one biological activity of the reference peptide. Homologs can include paralogs, orthologs, or naturally occurring alleles of the reference peptide. For example, the biological activity exhibited by obtustatin homologs is the inhibition of ally integrin-mediated cellular adhesion to collagen IV.

By “label” is meant any substance which can be incorporated into or conjugated to a compound, by chemical bonds or any other means, and which can be detected. By “libraries” is meant pools and subpools of pro-analogs.

“Peptide” and “protein” are used interchangeably, and refer to a compound comprised of amino acid residues covalently linked by peptide bonds. A protein or peptide must contain at least two amino acids and no limitation is placed on the maximum number of amino acids which can comprise a protein's or peptide's sequence. The amino acids of the peptides described herein and in the appended claims are understood to be either D or L amino acids with L amino acids being preferred.

“Pharmaceutically acceptable” means physiologically tolerable, for either human or veterinary application.

As used herein, “pharmaceutical compositions” include formulations for human and veterinary use.

“Pro-analogs” are compounds which can potentially be obtustatin or viperisrastatin analogs. Pro-analogs are regarded as analogs once it is determined that they exhibit one or more biological activities of obtustatin or viperisrastatin.

“RGD-independent integrins” are integrins which bind to amino acid sequences other than RGD.

“RGD-dependent integrins” are integrins which bind to the RGD amino acid sequence.

By “selectable label” is meant any substance which can be used to label the compounds of the invention, and which can then be used to selectively remove the labeled compound (and any material bound to the labeled compound) from a sample.

By “specifically bind,” as used to describe the interaction between an antibody and another molecule, is meant that the two bind to each other with greater affinity than to other, non-specific molecules.

As used herein, a peptide or a portion of a peptide which has a “substantially similar amino acid sequence” to a reference peptide means that the peptide, or a portion thereof, has an amino acid sequence identity or similarity to the reference peptide of greater than about 30%, preferably greater than about 60%, more preferably greater than about 70%, even more preferably greater than about 80%, and most preferably greater than about 90%. Amino acid sequence similarity or identity can be computed by using the BLASTP and TBLASTN programs which employ the BLAST (basic local alignment search tool) 2.0.14 algorithm; BLASTP and TBLASTN settings to be used in such computations are indicated in Table 1 below. Amino acid sequence identity is reported under “Identities” by the BLASTP and TBLASTN programs. Amino acid sequence similarity is reported under “Positives” by the BLASTP and TBLASTN programs. Techniques for computing amino acid sequence similarity or identity are within the skill in the art, and the use of the BLAST algorithm is described in Altschul et al., J. Mol. Biol. 215:403-10 (1990) and Altschul et al., Nucleic Acids Res. 25:3389-3402 (1997), the disclosures of which are herein incorporated by reference in their entirety. BLASTP and TBLASTN programs utilizing the BLAST 2.0.14 algorithm are available, for example, at the National Center for Biotechnology Information World Wide Web site BLAST server.

TABLE 1
Settings to be used for the computation of amino acid sequence
similarity or identity with BLASTP and TBLASTN programs
utilizing the BLAST 2.0.14 algorithm.

| | |
|---|---|
| Expect Value | 10 |
| Filter | Low complexity |
| | filtering using |
| | SEG program* |
| Substitution Matrix | BLOSUM62 |
| Gap existence cost | 11 |
| Per residue gap cost | 1 |
| Lambda ratio | 0.85 |
| Word size | 3 |

*The SEG program is described by Wootton and Federhen, Comput. Chem. 17: 149-163 (1993).

“Substantially purified” refers to a population of peptides or cells which is substantially homogenous in character due to the removal of other compounds (e.g., other peptides, nucleic acids, carbohydrates, lipids) or other cells originally present. “Substantially purified” is not meant to exclude artificial or synthetic mixtures with other compounds, or the presence of impurities which do not interfere with biological activity, and which can be present, for example, due to incomplete purification, addition of stabilizers, or formulation into a pharmaceutically acceptable preparation.

“Synthetic mutant” includes any purposefully generated mutant derived from obtustatin or viperisrastatin. Such mutants can be generated by, for example, chemical mutagenesis, polymerase chain reaction (PCR) based approaches, or primer based mutagenesis strategies within the skill in the art.

### BRIEF DESCRIPTION OF THE FIGURES

FIG. 1 is the elution profile for Viper lebetina obtusa venom proteins applied to a C₁₈ reverse phase HPLC chromatography column running a linear 0-80% acetonitrile in H₂O gradient for 45 minutes. The fraction containing obtustatin elutes at approximately 21 minutes. After elution the obtustatin containing fraction was lyophylized. Protein elution was followed using A_{206 nm}.

FIG. 2 is the elution profile for Viper lebetina obtusa venom proteins present in the lyophylized, obtustatin-containing fraction collected as described in FIG. 1. The lyophylized proteins were resuspended in trifluoroacetic acid and applied to a C₁₈ reverse phase HPLC chromatography column running a linear 20-70% acetonitrile in H₂O gradient for 70 minutes. Obtustatin elutes at approximately 23 minutes. Protein elution was followed using A_{206 nm}.

FIG. 3 is a comparison of the obtustatin and viperisrastatin amino acid sequences with the short disintegrins echistatin, eristostatin and ocellatin. Conserved cysteine residues are underlined, the KTS and RGD binding sites are italicized, and the amino acid differences between obtustatin and viperisrastatin are shown in bold.

FIG. 4 shows the effect of obtustatin, EP-obtustatin (an ethylpyridylated derivative of obtustatin) and fragments of obtustatin on the α1β1 integrin-mediated adhesion of K562 cells to collagen IV. Filled circles represent native obtustatin peptide sequence:

CTTGPCCRQCKLKPAGTTCWKTSLTSHYCTGKSCDCPLYPG (SEQ ID NO: 1). Open circles represent EP-obtustatin. Filled triangles represent peptide sequence CWKTSLTSHYC (SEQ ID NO: 5). Open triangles represent peptide sequence TSLTS (SEQ ID NO: 6). Filled squares represent peptide sequence CKLKPAGTTC (SEQ ID NO: 7).

FIG. 5 shows the effect of obtustatin on adhesion of MV3 cells to immobilized collagen IV. Filled circles show the effect of obtustatin on α1β1 and α2β1 integrin-mediated binding to collagen IV; no blocking antibodies are present. Open circles show the effects of obtustatin on α2β1 binding when the α1β1 integrin is blocked by the α1 integrin subunit specific AJH10 antibody. Filled triangles show the effect of obtustatin binding on α1β1 integrin binding when the α2β1 integrin is blocked by the α2 integrin subunit specific P1E6 antibody.

FIG. 6 shows the effect of obtustatin and eristostatin on angiogenesis in a Japanese quail chorioallantoic membrane (CAM) assay. Dᵣ represents fractal dimension as measure of space-filling branching pattern for skeletonized images. Bar A: PBS (control); bar B: eristostatin (20 μg); bar C: eristostatin (50 μg); bar D: obtustatin (20 μg); bar E: obtustatin (50 μg).

FIG. 7 shows the effect of single amino acid changes within the peptide sequence CWKTSLTSHYC (SEQ ID NO: 5) on adhesion of α1K562 cells to collagen IV.

FIG. 8 is a stereo plot of the best three-dimensional structures of obtustatin (1MPZ) superimposed over the backbone of well-defined residues. (A) only backbone atoms are displayed; (B) backbone and heavy atoms are shown.

FIG. 9 is a plot of average tumor size vs. days in C57BL/6 mice implanted with Lewis lung carcinoma cells, and injected with 5 mg/kg obtustatin (triangles), 5 mg/kg echistatin (squares), or PBS (control group; circles). Tumor volume was measured using the standard formula length×width²×0.52. N=4 per group; bars represent standard error.

FIG. 10 is a histogram showing caspase-3 activity in (A) microvascular endothelial cells, and (B) human melanoma MV3 cells treated with vehicle (control), TNF-α, vincristine or obtustatin. The filled bars represent unstimulated cells and the open bars represent cells stimulated with VEGF/bFGF in a ratio of 5 ng/3 ng per ml.

FIG. 11 is a histogram showing BrdU incorporation into (A) human microvascular endothelial cells (HMVEC) and (B) melanoma cells (HS.939T) treated with vehicle (control), 2 μM obtustatin, 2 μM echistatin or 50 ng/ml TNF-α.

FIG. 12 is a plot of fluorescence units vs. obtustatin concentration showing the extent of MV3 melanoma cell migration through HUVECs in the presence of varying concentrations of obtustatin. Open circles represent MV3 cells treated with obtustatin using collagen IV as a chemoattractant. Open triangles represent MV3 cells treated with obtustatin using FGF as a chemoattractant. MV3 cells were also treated with a random control peptide, using collagen IV (closed circles) or FGF (closed triangles) as chemoattractants.

### DETAILED DESCRIPTION OF THE INVENTION

The present invention provides a novel family of small non-RGD disintegrins, which containing the tripeptide binding sequence lysine-threonine-serine (KTS). The KTS-containing disintegrins inhibit the binding of α1β1 integrin to its adhesive ligand collagen IV. Two disintegrins in this family, called “obtustatin” and “viperisrastatin,” are also provided.

The biological activity of KTS-disintegrins can be measured by cell adhesion assays or epitope exposure assays capable of detecting α1β1 integrin binding activity, as are known in the art. See, for example, Marcinkiewicz et al., Biochem. J. 317: 817-825 (1996), the entire disclosure of which is herein incorporated by reference, and the cell adhesion assays presented in Examples 4 and 5. Inhibition of α1β1 integrin activity exhibited by the present KTS-disintegrins can be expressed as an IC₅₀ value. The IC₅₀ value is the concentration of a KTS-disintegrin which inhibits 50% of the activity level of an α1β1 integrin which was measured in the absence of the KTS-disintegrin.

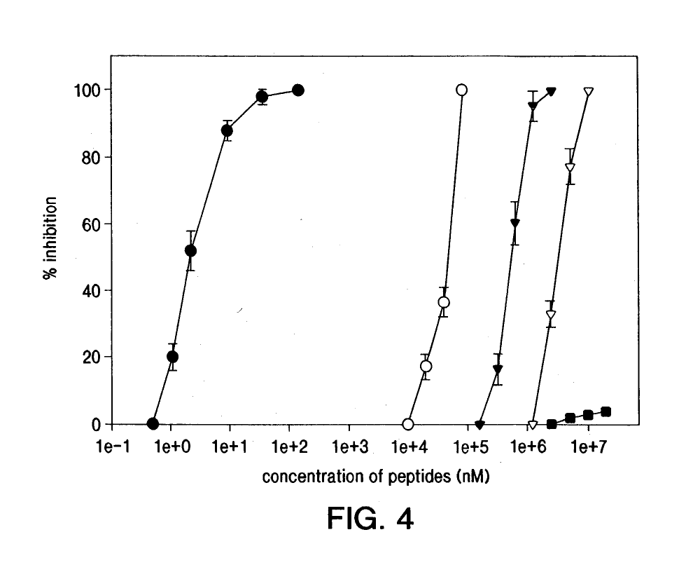

KTS-disintegrins preferably have an IC₅₀ value between 1 pM and 1 M, more preferably between 0.01 nM and 10 nM, and most preferably between 1 nM and 3 nM. For example, the in vitro IC₅₀ of native obtustatin as measured by the cell adhesion assay of Example 6 is 2 nM (see FIG. 4 and Table 5), and the in vitro IC₅₀ of native viperisrastatin is 0.08 nM (see Table 6). EP-obtustatin, in which the cysteine thiol groups of native obtustatin have been alkylated with 4-vinylpyridine, has an IC₅₀ value of 30 μM.

Obtustatin is a peptide of 41 amino acid residues, which contains eight conserved cysteine residues. The primary amino acid sequence of obtustatin as determined by automated Edman degradation is:

CTTGPCCRQCKLKPAGTTCWKTSLTSHYCTGKSCDCPLYPG (SEQ ID NO: 1).

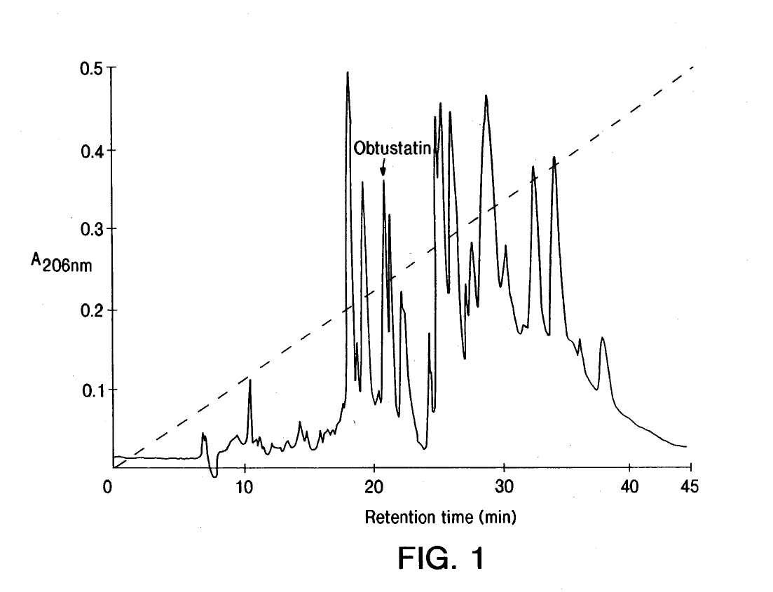

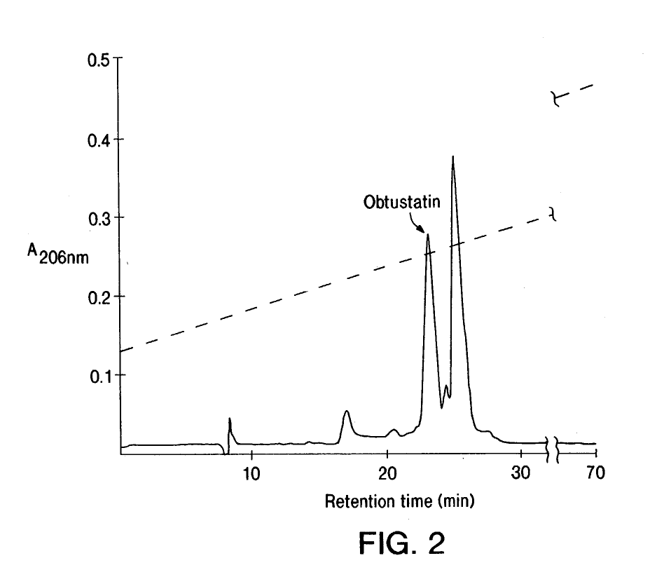

The KTS binding site is italicized, and the conserved cysteines are underlined. Obtustatin was substantially purified from the venom of the viper Vipera lebetina obtusa, and has an apparent molecular weight of 4395 daltons as determined by mass spectroscopy. Obtustatin elutes at approximately 21 minutes from a C₁₈ HPLC reverse phase chromatography column running a linear 0-80% acetonitrile in H₂O gradient for 45 minutes (FIG. 1) and at approximately 23 minutes from the same column running a linear 20-70% acetonitrile in H₂O gradient for 70 minutes (FIG. 2).

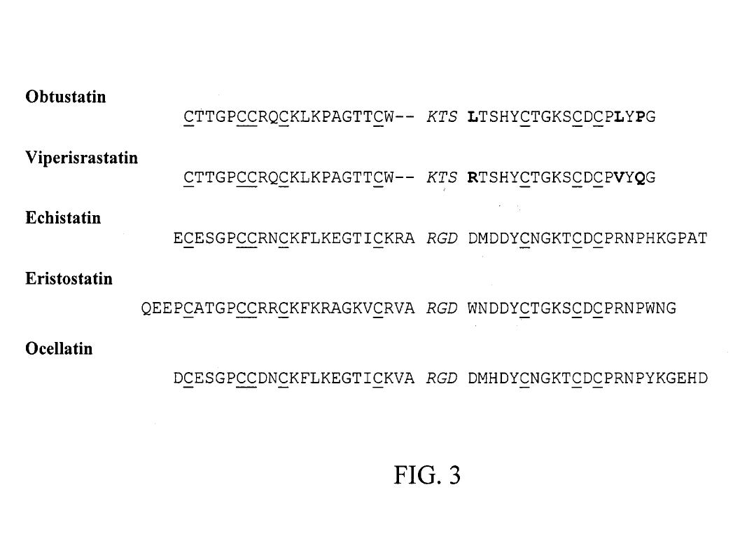

The carboxy-terminal portion of obtustatin is unconserved relative to eristostatin and echistatin (FIG. 3). The carboxy-terminal amino acid sequences of echistatin and eristostatin appear to be involved in the selectivity of these disintegrins for αIIbβ3 integrin. Without wishing to be bound by any theory, the differences in the carboxy-terminus of obtustatin relative to echistatin and eristostatin might explain the different integrin-binding specificity of obtustatin.

Viperisrastatin is a peptide of 41 amino acid residues, which contains eight conserved cysteine residues and has a sequence which differs from obtustatin by only three amino acids. Viperisrastatin was substantially purified from the venom of the viper Vipera palestinae under the same conditions used to purify obtustatin described above. The primary amino acid sequence of viperisrastatin as determined by automated Edman degradation is:

CTTGPCCRQCKLKPAGTTCWKTSRTSHYCTGKSCDCPVYQG (SEQ ID NO: 2).

The KTS binding site is italicized, and the conserved cysteines are underlined. The residues which differ from obtustatin are shown in bold. Like obtustatin, the carboxy-terminal portion of viperisrastatin is unconserved relative to eristostatin and echistatin (FIG. 3).

The ability of viperisrastatin to inhibit the activity of human α1β1 integrin is approximately two orders of magnitude greater than obtustatin, whereas both KTS-containing disintegrins showed similar ability to inhibit activity of mouse α1β1 integrin; see Table 6 and Example 14 below. Without wishing to be bound by any theory, the selectivity of viperisrastatin for human α1β1 integrin is likely related to the arginine residue immediately following the KTS motif in the C-terminal direction. In obtustatin, there is a leucine in this position; see FIG. 3 and SEQ ID NOS. 1 and 2 above. It is known from research into the RGD-disintegrins that the amino acid adjacent to RGD motif in the C-terminal direction is important for potency and selectivity of the disintegrin. Again without wishing to be bound by any theory, the amino acid residue immediately adjacent to the KTS motif in the C-terminal direction in KTS-containing disintegrins also appears to affect their potency and selectivity.

The invention thus provides a substantially purified obtustatin or viperisrastatin, or a biologically active fragment thereof, which inhibits α1β1 integrin binding to collagen IV. Preferably, the biologically active obtustatin or viperisrastatin fragments are 5-30 amino acids in length, although smaller fragments comprising from 5-25 or 5-20 amino acids are also contemplated.

The invention also provides KTS-disintegrins comprising the sequence X₁-Lys-Thr-Ser-X₂, wherein X₁ is from zero to twenty-five amino acids and X₂ is from zero to twenty amino acids. In one embodiment, X₁ can be zero amino acids, or can comprise the segment Cys-Thr-Thr-Gly-Pro-Cys-Cys-Arg-Gln-Cys-Lys-Leu-Lys-Pro-Ala-Gly-Thr-Thr-Cys-Trp (SEQ ID NO: 3) or an amino-terminal truncation fragment thereof containing at least one amino acid. In this same embodiment, X₂ can be zero amino acids, or can comprise the segment X₄-Thr-Ser-His-Tyr-Cys-Thr-Gly-Lys-Ser-Cys-Asp-Cys-Pro-X₅-Tyr-X₆-Gly (SEQ ID NO: 4), or a carboxy-terminal truncation fragment thereof containing at least one amino acid. In this segment, X₄ can be Leu or Arg, provided that when X₄ is Leu then X₅ is Leu and X₆ is Pro, and when X₄ is Arg then X₅ is Val and X₆ is Gln.

The N-terminal truncation fragments comprising X₁ can be formed by the sequential removal of amino acids from the N-terminus of SEQ ID NO:3. These truncation fragments are given in Table 2 below.

TABLE 2
SEQ ID NO: 3 and N-Terminal Truncation Fragments
Of SEQ ID NO: 3

| Fragment | Sequence | SEQ ID NO. |
|---|---|---|
| A | CTTGPCCRQCKLKPAGTTCW | 3 |
| B | TTGPCCRQCKLKPAGTTCW | 8 |
| C | TGPCCRQCKLKPAGTTCW | 9 |
| D | GPCCRQCKLKPAGTTCW | 10 |
| E | PCCRQCKLKPAGTTCW | 11 |
| F | CCRQCKLKPAGTTCW | 12 |
| G | CRQCKLKPAGTTCW | 13 |
| H | RQCKLKPAGTTCW | 14 |
| I | QCKLKPAGTTCW | 15 |
| J | CKLKPAGTTCW | 16 |
| K | KLKPAGTTCW | 17 |
| L | LKPAGTTCW | 18 |
| M | KPAGTTCW | 19 |
| N | PAGTTCW | 20 |
| O | AGTTCW | 21 |
| P | GTTCW | 22 |
| Q | TTCW | 23 |
| R | TCW | |
| S | CW | |
| T | W | |

The C-terminal truncation fragments comprising X₂ can be formed by the sequential removal of amino acids from the C-terminus of SEQ ID NO: 4; these truncation fragments are given in Table 3 below.

TABLE 3
SEQ ID NO: 4 and C-Terminal Truncation Fragments
of SEQ ID NO: 4

| Fragment | Sequence | SEQ ID NO. |
|---|---|---|
| 1 | X₄TSHYCTGKSCDCPX₅YX₆G | 4 |
| 2 | X₄TSHYCTGKSCDCPX₅YX⁶ | 24 |
| 3 | X₄TSHYCTGKSCDCPX₅Y | 25 |
| 4 | X₄TSHYCTGKSCDCPX₅ | 26 |
| 5 | X₄TSHYCTGKSCDCP | 27 |
| 6 | X₄TSHYCTGKSCDC | 28 |
| 7 | X₄TSHYCTGKSCD | 29 |
| 8 | X₄TSHYCTGKSC | 30 |
| 9 | X₄TSHYCTGKS | 31 |
| 10 | X₄TSHYCTGK | 32 |
| 11 | X₄TSHYCTG | 33 |
| 12 | X₄TSHYCT | 34 |
| 13 | X₄TSHYC | 35 |
| 14 | X₄TSHY | 36 |
| 15 | X₄TSH | 37 |
| 16 | X₄TS | |
| 17 | X₄T | |
| 18 | X₄ | |

Biologically active compounds of the formula X₁-KTS-X₂ are made by combining SEQ ID NO: 3 or fragments of SEQ ID NO: 3 from Table 2 (representing X₁) and SEQ ID NO: 4 fragments of SEQ ID NO: 4 from Table 3 (representing X₂) with the core sequence KTS. It is understood that all possible combinations of the sequences listed in Tables 2 and 3 above with the KTS core sequence are contemplated as being part of the present invention.

These combinations are shown in Table 4 as follows: SEQ ID NO: 3 and its N-terminal truncation fragments from Table 2, represented by letters A through T, are listed in the leftmost column of Table 4. SEQ ID NO: 4 and its C-terminal truncation fragments from Table 3, represented by the numerals 1 through 17, are listed in the topmost row of Table 4. Biologically active fragments are identified by creating a matrix of N-terminal fragments and C-terminal fragments as seen in Table 4. It is understood that SEQ ID NO: 3 or the N-terminal fragments are attached to the K residue of the core sequence KTS, and SEQ ID NO: 4 or the C-terminal fragments are attached to the S residue of the core sequence KTS. For ease of illustration, the core sequence KTS is not represented in the Table 4 matrix.

TABLE 4
Matrix of Biologically Active Peptides From All Possible
Combinations of Sequences From Tables 2 and 3

| Frag | 1 | 2 | 3 | 4 | 5 | 6 | 7 | 8 | 9 | 10 | 11 | 12 | 13 | 14 | 15 | 16 | 17 | 18 |
|---|---|---|---|---|---|---|---|---|---|---|---|---|---|---|---|---|---|---|
| A | A1 | A2 | A3 | A4 | A5 | A6 | A7 | A8 | A9 | A10 | A11 | A12 | A13 | A14 | A15 | A16 | A17 | A18 |
| B | B1 | B2 | B3 | B4 | B5 | B6 | B7 | B8 | B9 | B10 | B11 | B12 | B13 | B14 | B15 | B16 | B17 | B18 |
| C | C1 | C2 | C3 | C4 | C5 | C6 | C7 | C8 | C9 | C10 | C11 | C12 | C13 | C14 | C15 | C16 | C17 | C18 |
| D | D1 | D2 | D3 | D4 | D5 | D6 | D7 | D8 | D9 | D10 | D11 | D12 | D13 | D14 | D15 | D16 | D17 | D18 |
| E | E1 | E2 | E3 | E4 | E5 | E6 | E7 | E8 | E9 | E10 | E11 | E12 | E13 | E14 | E15 | E16 | E17 | E18 |
| F | F1 | F2 | F3 | F4 | F5 | F6 | F7 | F8 | F9 | F10 | F11 | F12 | F13 | F14 | F15 | F16 | F17 | F18 |
| G | G1 | G2 | G3 | G4 | G5 | G6 | G7 | G8 | G9 | G10 | G11 | G12 | G13 | G14 | G15 | G16 | G17 | G18 |
| H | H1 | H2 | H3 | H4 | H5 | H6 | H7 | H8 | H9 | H10 | H11 | H12 | H13 | H14 | H15 | H16 | H17 | H18 |
| I | I1 | I2 | I3 | I4 | I5 | I6 | I7 | I8 | I9 | I10 | I11 | I12 | I13 | I14 | I15 | I16 | I17 | I18 |
| J | J1 | J2 | J3 | J4 | J5 | J6 | J7 | J8 | J9 | J10 | J11 | J12 | J13 | J14 | J15 | J16 | J17 | J18 |
| K | K1 | K2 | K3 | K4 | K5 | K6 | K7 | K8 | K9 | K10 | K11 | K12 | K13 | K14 | K15 | K16 | K17 | K18 |
| L | L1 | L2 | L3 | L4 | L5 | L6 | L7 | L8 | L9 | L10 | L11 | L12 | L13 | L14 | L15 | L16 | L17 | L18 |
| M | M1 | M2 | M3 | M4 | M5 | M6 | M7 | M8 | M9 | M10 | M11 | M12 | M13 | M14 | M15 | M16 | M17 | M18 |
| N | N1 | N2 | N3 | N4 | N5 | N6 | N7 | N8 | N9 | N10 | N11 | N12 | N13 | N14 | N15 | N16 | N17 | N18 |
| O | O1 | O2 | O3 | O4 | O5 | O6 | O7 | O8 | O9 | O10 | O11 | O12 | O13 | O14 | O15 | O16 | O17 | O18 |
| P | P1 | P2 | P3 | P4 | P5 | P6 | P7 | P8 | P9 | P10 | P11 | P12 | P13 | P14 | P15 | P16 | P17 | P18 |
| Q | Q1 | Q2 | Q3 | Q4 | Q5 | Q6 | Q7 | Q8 | Q9 | Q10 | Q11 | Q12 | Q13 | Q14 | Q15 | Q16 | Q17 | Q18 |
| R | R1 | R2 | R3 | R4 | R5 | R6 | R7 | R8 | R9 | R10 | R11 | R12 | R13 | R14 | R15 | R16 | R17 | R18 |
| S | S1 | S2 | S3 | S4 | S5 | S6 | S7 | S8 | S9 | S10 | S11 | S12 | S13 | S14 | S15 | S16 | S17 | S18 |
| T | T1 | T2 | T3 | T4 | T5 | T6 | T7 | T8 | T9 | T10 | T11 | T12 | T13 | T14 | T15 | T16 | T17 | T18 |

Thus, referring to Table 4, biologically active peptide M10 is formed from N-terminal fragment M (KPAGTTCW; SEQ ID NO: 19) and C-terminal fragment 10 (X₄TSHYCTGK, SEQ ID NO: 32, where X₄ is L) attached to the core sequence KTS, to give KPAGTTCWKTSLTSHYCTGK (SEQ ID NO: 38). Likewise, biologically active peptide S14 is formed from N-terminal fragment S (CW) and C-terminal fragment 14 (X₄TSHY, SEQ ID NO: 36, where X₄ is L) attached to the core sequence KTS, to give CWKTSLTSHY (SEQ ID NO: 39). Biologically active peptide F4 is formed from N-terminal fragment F (CCRQCKLKPAGTTCW; SEQ ID NO: 12) and C-terminal fragment 4 (X₄TSHYCTGKSCDCPX₅, SEQ ID NO: 26, where X₄ is R and X₅ is Val) attached to core sequence KTS, to give CCRQCKLKPAGTTCWKTSRTSHYCTGKSCDCPV (SEQ ID NO: 40). It is apparent that all combinations of biologically active peptides can be identified by reference to Tables 2, 3 and 4.

As shown in FIG. 3, the KTS-disintegrins have a conserved cysteine skeleton relative to each other, and relative to short RGD-disintegrins. The cysteine skeleton is known to be important for maintaining the three-dimensional structure necessary for binding of disintegrins to their target ligands. The invention thus provides KTS-disintegrins comprising the sequence Cys-Xaa-Xaa-Xaa-Xaa-Cys-Cys-Xaa-Xaa-Cys-Xaa-Xaa-Xaa-Xaa-Xaa-Xaa-Xaa-Xaa-Cys-Xaa-Lys-Thr-Ser-Xaa-Xaa-Xaa-Xaa-Xaa—Cys-Xaa-Xaa-Xaa-Xaa-Cys-Xaa-Cys-X₇ (SEQ ID NO: 41), where X₇ is zero or any 1, 2, 3, 4, or 5 amino acids, and each Xaa is independently any amino acid.

The KTS disintegrins, including obtustatin and viperisrastatin, and biologically active fragments thereof, can comprise natural or synthetic peptides produced by any known means, including synthesis by biological or chemical methods. Biological synthesis of peptides is well known in the art, and includes the transcription and translation of a synthetic genes encoding a given peptide. Chemical peptide synthesis includes manual and automated techniques well known to those skilled in the art. For example, automated synthesis can be performed with commercially available peptide synthesizers. Biologically active fragments according to the invention can also be obtained by the digestion or fragmentation of larger natural or synthetic peptides. Techniques to synthesize or otherwise obtain peptides and peptide fragments are well known in the art.

The peptides and fragments of the present invention can be synthesized de novo using conventional solid phase synthesis methods. In such methods, the peptide chain is prepared by a series of coupling reactions in which the constituent amino acids are added to the growing peptide chain in the desired sequence. The use of various N-protecting groups, e.g., the carbobenzyloxy group or the t-butyloxycarbonyl group; various coupling reagents e.g., dicyclohexylcarbodiimide or carbonyldimidazole; various active esters, e.g., esters of N-hydroxyphthalimide or N-hydroxy-succinimide; and the various cleavage reagents, e.g., trifluoroactetic acid (TFA), HCl in dioxane, boron tris-(trifluoracetate) and cyanogen bromide; and reaction in solution with isolation and purification of intermediates are within the skill in the art. A preferred peptide synthesis method follows conventional Merrifield solid phase procedures well known to those skilled in the art.

Additional information about solid phase synthesis procedures can be had by reference to Steward and Young, Solid Phase Peptide Synthesis, W. H. Freeman & Co., San Francisco, 1969; the review chapter by Merrifield in Advances in Enzymology 32: 221-296, F. F. Nold, Ed., Interscience Publishers, New York, 1969; and Erickson and Merrifield, The Proteins 2 :61-64 (1990), the entire disclosures of which are incorporated herein by reference. Crude peptide preparations resulting from solid phase syntheses can be purified by methods well known in the art, such as preparative HPLC. The amino-terminus of peptides undergoing synthesis can be protected according to the methods described for example by Yang et al., FEBS Lett. 272 :61-64 (1990), the entire disclosure of which is herein incorporated by reference.

The invention also provides compounds comprising biologically active derivatives of obtustatin. Techniques for obtaining such derivatives are with the skill in the art and include, for example, standard recombinant nucleic acid techniques, solid phase peptide synthesis techniques, and chemical synthetic techniques as described above. Derivatives according to the invention can also be produced by using linking groups to join or replace portions of, for example, obtustatin or viperisrastatin. Suitable linking groups include cyclic compounds capable of connecting an amino-terminal portion and a carboxyl terminal portion of one or more peptides. Techniques for generating peptide derivatives are also described in U.S. Pat. No. 6,030,942 the entire disclosure of which is herein incorporated by reference (derivatives are designated “peptoids” in the U.S. Pat. No. 6,030,942 patent). Derivatives can also incorporate labels such as radioisotopes into their structure and can be in the form of salts such as pharmaceutically acceptable salts.

Examples of obtustatin derivatives include synthetic mutants of these peptides. Derivatives of obtustatin or viperisrastatin can also include, for example, fusion peptides in which a portion of the fusion peptide has a substantially similar amino acid sequence to obtustatin or viperisrastatin. Fusion peptides can be generated by any means which permits linking two or more peptide sequences including, for example, standard recombinant nucleic acid techniques, solid phase peptide synthesis techniques, or other techniques which are well known to those skilled in the art.

The invention also provides compounds comprising biologically active homologs of obtustatin. For example, biologically active homologs of obtustatin have substantially similar amino acid sequence to obtustatin and inhibit binding of α1β1 integrin to collagen IV, and can be identified on this basis. It is particularly preferred that homologs of obtustatin contain the core amino acid sequences KTSLT, SL, SLT or KTS. Based on the sequence and activity data presented herein, viperisrastatin can be considered a biologically active homolog of obtustatin.

The invention also provides compounds comprising biologically active analogs of obtustatin. These analogs can, for example, be small organic molecules capable of inhibiting α1β1 integrin activity. Analogs can incorporate labels such as radioisotopes into their structure and can be in the form of salts such as pharmaceutically acceptable salts.

Without wishing to be bound by any theory, it is believed that biologically active KTS-disintegrin analogs comprise a structure called a pharmacophore, which mimics the physico-chemical and spatial characteristics of the KTS binding site. Consequently, pro-analogs can be designed based on variations in the molecular structure of the α1β1 integrin with which the KTS binding site interacts, or can be designed based on portions of obtustatin or viperisrastatin.

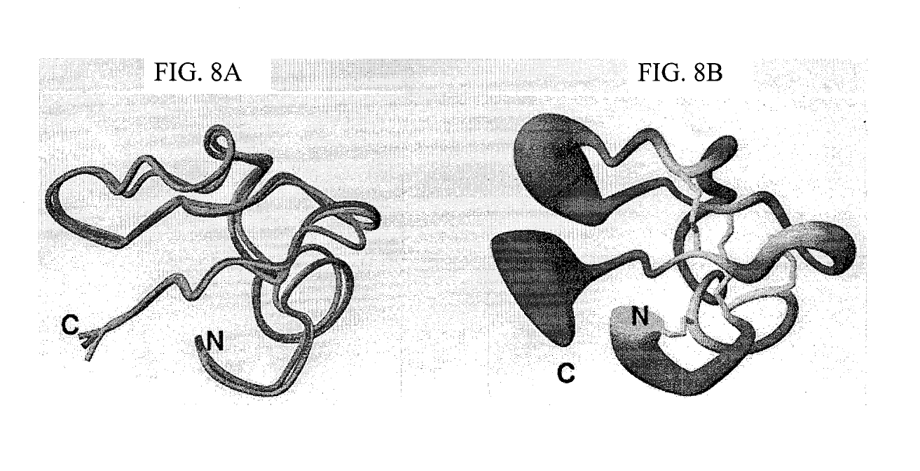

The structure of the various portions of obtustatin, viperisrastatin, or α1β1 integrin can be determined, for example, using well-known NMR (nuclear magnetic resonance), crystallographic, or computational methods which permit the electron density, electrostatic charges or molecular structure of these peptides to be mapped; these methods are well known to those skilled in the art. For example, the three-dimensional structure of obtustatin, based on NMR mapping studies of the molecule, is presented in FIGS. 8A-8B.

Alternatively, pro-analogs of the KTS-disintegrins can be designed by using the retrosynthetic, target oriented, or diversity-oriented synthesis strategies described by Schreiber, Science 287:1964-1969 (2000) the entire disclosure of which is herein incorporated by reference. Retrosynthetic strategies require that key structural elements in a molecule (such as obtustatin or viperisrastatin) which interacts with a target molecule (such as the α1 β1 integrin) be identified and then incorporated into the structure of otherwise distinct pro-analogs generated by organic syntheses. U.S. Pat. No. 6,030,942, in particular Example 4 therein, describes retrosynthetic methods for the design and selection of analogs based on key structural elements in an inhibitory peptide and is incorporated herein in its entirety (analogs are designated “peptidomimetics” in the U.S. Pat. No.6,030,942 patent).

The solid-phase synthesis methods described by Schreiber supra can be used to generate a library of distinct pro-analogs generated by organic syntheses. Briefly, a suitable synthesis support (e.g., a resin) is coupled to a pro-analog precursor and the pro-analog precursor is subsequently modified by organic reactions such as Diels-Alder cyclization. The immobilized pro-analog can then be released from the solid substrate.

Pools and subpools of pro-analogs can be generated by automated synthesis techniques in parallel, such that all synthesis and resynthesis can be performed in a matter of days; pools and subpools of pro-analogs are said to comprise libraries. Once generated, pro-analog libraries can be screened for analogs; i.e. compounds exhibiting one or more biological activities of a KTS-disintegrin; e.g., obtustatin or viperisrastatin. KTS-disintegrin analogs can be identified, for example, by automated screening assays performed in 96-well plates in which the ability of one or more pro-analogs present in solution to inhibit α1β1 integrin activity is assayed via a cell adhesion assay of the type described in Example 4.

The KTS disintegrins, including obtustatin and viperisrastatin, and biologically active fragments, derivative, homologs and analogs thereof (also referred to herein as “compounds of the invention”) can be modified with other substances. Modification of the compounds of the invention can alter their activity, for example by altering characteristics such as in vivo tissue partitioning, peptide degradation rate, integrin binding or integrin specificity. The modifications can also confer additional characteristics to the compounds of the invention, such as the ability to be detected, manipulated or targeted. The modifying substance can be joined to the compounds of the invention, for example, by chemical means (e.g., by covalent bond, electrostatic interaction, Van der Waals forces, hydrogen bond, ionic bond, chelation, and the like) or by physical entrapment.

For example, the compounds of the invention can be modified with a label. Suitable labels include substances which are: magnetic resonance active; radiodense; fluorescent; radioactive; detectable by ultrasound; detectable by visible, infrared or ultraviolet light. Preferred labels include fluorescein isothiocyanate, peptide chromophores such as phycoerythrin or phycocyanin and the like; bioluminescent peptides such as the luciferases originating from Photinus pyrali; and fluorescent proteins originating from Renilla reniformi.

The compounds of the invention can also be modified with polymeric and macromolecular structures (e.g., liposomes, zeolites, dendrimers, magnetic particles, and metallic beads) or targeting groups (e.g., signal peptide sequences, ligands, lectins, or antibodies). Peptides or peptide fragments of the invention can further be modified with carboxyl or amino-terminal protecting groups, amino-acid side chain modifying groups, and the like.

Methods of modifying compounds of the invention with other substances are well known to those skilled in the art. For example, methods of conjugating fluorescent compounds such as fluorescein isothiocyanate to the short disintegrin eristostatin are described in Danen et al., Exp. Cell Res., 238: 188-86 (1998), the entire disclosure of which is incorporated herein by reference. Methods of radiolabeling peptides with ¹²⁵I are disclosed by Sambrook et al. in Molecular Cloning: A Laboratory Manual, Cold Spring Harbor Laboratories, Second Ed., (1989), the disclosure of which is incorporated herein by reference.

The invention also provides a method of inhibiting α1β1 integrin from binding to their adhesive ligands. The α1β1 integrin can be free or bound to a cell membrane. Generally, inhibition of α1β1 integrin binding where α1β1 integrin is free will be performed in vitro. The inhibition of α1β1 integrin binding where α1β1 integrin is bound to a cell membrane can be performed in vitro or in vivo. The method comprises contacting a sample with an effective amount of one or more KTS disintegrins, or biologically active fragments, derivative, homologs and analogs thereof, so that the binding of α1β1 integrin to its adhesive ligand is inhibited.

An effective amount of a KTS disintegrin, or a biologically active fragment, derivative, homolog or analogs thereof, sufficient to prevent binding of the integrin to a ligand can be determined by cell adhesion assays or epitope exposure assays, as discussed above and as shown in Example 4.

The compounds of the invention can be administered in vivo to a subject suffering from a disease or biological condition associated with the binding of α1β1 integrin to its adhesive ligand. The subject can be any animal, preferably a mammal, and most preferably a human being. Diseases associated with the binding of α1β1 integrin to its adhesive ligand include insulin dependent diabetes mellitus, multiple sclerosis, rheumatoid arthritis, ulcerative colitis, arteriosclerosis, and cancer. Biological conditions associated with the binding of α1β1 integrin to its adhesive ligand include asthma, allergy, organ rejection, and restenosis of arteries after surgery or angioplasty, and angiogenesis.

For example, KTS disintegrins, and biologically active fragments, derivatives, homologs and analogs thereof, are useful in the treatment of cancers which express α1β1 integrins on the surface of the cancer or tumor cells. Such cancers include, for example, leukemias, melanomas, lymphomas, and sarcomas.

Moreover, the α1β1 integrin is known to be involved in angiogenesis and neovascularization, two biological processes which occur during tumorigenesis. Inhibition of α1β1 containing integrins at tumor sites with the compounds of the invention can therefore limit tumor growth, metastasis, and vascularization regardless of whether the tumor cells express α1β1 integrin.

The KTS disintegrins, and biologically active fragments, derivative, homologs and analogs thereof, are also useful in the treatment of angiogenesis-mediated disorders other than cancer. Such disorders include metastasis, comeal graft rejection, ocular neovascularization, retinal neovascularization, diabetic retinopathy, retrolental fibroplasia, neovascular glaucoma, gastric ulcer, infantile hemangiomas, angiofibroma of the nasopharynx, avascular necrosis of bone, and endometriosis. Thus, a method for treating, inhibiting or delaying the onset of an angiogenesis-mediated disorder in a subject is provided, comprising administering to a subject in need of such treatment an effective amount of a KTS disintegrin, or a biologically active fragment, derivative, homolog or analogs thereof.

The α1β1 integrin is also involved in wound healing and blood clot formation. Inhibition of α1β1 integrin binding can therefore be used to control thrombic occlusion formation, blood clot formation and wound healing. The invention thus provides a method of treating diseases or biological conditions associated with the binding of α1β1 integrin to its adhesive ligand, comprising administering to a subject an amount of a KTS disintegrin, or a biologically active fragment, derivative, homolog or analog thereof, sufficient to inhibit the binding of α1β1 integrin with its adhesive ligand. In preferred embodiments, the compounds of the invention are administered as pharmaceutically acceptable salts or a pharmaceutical composition.

Generally, the amount of the present compounds administered to a subject in vivo depends upon the degree of integrin inhibition that is desired. Those skilled in the art can derive appropriate dosages and schedules of administration to suit the specific circumstances and needs of the patient. For example, suitable doses of a KTS disintegrin, or a biologically active fragment, derivative, homolog or analog thereof, to be administered can be estimated from the cell adhesion assays or epitope exposure assays discussed above. Typically, dosages are between about 0.001 mg/kg and about 100 mg/kg body weight, preferably between about 0.01 mg/kg and about 10 mg/kg body weight, and particularly preferably between about 0.05 mg/kg and about 5 mg/kg body weight.

For in vivo applications, the compounds of the invention can comprise a pharmaceutically acceptable salt. Suitable acids which are capable of forming such salts include inorganic acids such as hydrochloric acid, hydrobromic acid, perchloric acid, nitric acid, thiocyanic acid, sulfuric acid, phosphoric acid and the like; and

organic acids such as formic acid, acetic acid, propionic acid, glycolic acid, lactic acid, anthranilic acid, cinnamic acid, naphthalene sulfonic acid, sulfanilic acid and the like.

When used in vivo, the compounds of the invention are preferably administered as a pharmaceutical composition. The invention thus provides pharmaceutical compositions comprising a KTS disintegrin, or a biologically active fragment, derivative, homolog or analog thereof, or a pharmaceutically acceptable salt of these compounds, and a pharmaceutically acceptable carrier.

Pharmaceutically acceptable carriers include physiologically tolerable or acceptable diluents, excipients, solvents or adjuvants. The compositions are preferably sterile and nonpyrogenic. Examples of suitable carriers include, but are not limited to, water, normal saline, dextrose, mannitol, lactose or other sugars, lecithin, albumin, sodium glutamate cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary substances or excipients such as wetting agents, emulsifying agents, pH buffering agents, antibacterial and antifungal agents (such as parabens, chlorobutanol, phenol, sorbic acid, and the like). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the present invention can be prepared in a manner fully within the skill of the art.

The compounds of the invention or pharmaceutical compositions comprising these compounds can be administered by any method designed to expose α1β1 integrin expressing cells of a subject to the compounds, so that the compounds can have a physiological effect. Administration can occur enterally or parenterally; for example orally, rectally, intracistemally, intravaginally, intraperitoneally, locally (e.g., with powders, ointments or drops), or as a buccal or nasal spray or aerosol. Parenteral administration is preferred. Particularly preferred parenteral administration methods include intravascular administration (e.g. intravenous bolus injection, intravenous infusion, intra-arterial bolus injection, intra-arterial infusion and catheter instillation into the vasculature), peri- and intra-target tissue injection (e.g. peri-tumoral and intra-tumoral injection), subcutaneous injection or deposition including subcutaneous infusion (such as by osmotic pumps) and direct application to the target area, for example by a catheter or other placement device. For example, if a subject is being treated for restenosis of coronary arteries after balloon angioplasty, the α1β1 integrin-inhibiting compounds can be administered by intra-arterial infusion at the site of catheterization.

Where the administration of the compounds of the invention, or pharmaceutical compositions thereof, is by injection or direct application, the injection can be in a single dose or in multiple doses. Where the administration is by infusion, the infusion can be a single sustained dose over a prolonged period of time or multiple infusions.

The a KTS disintegrins, or a biologically active fragments, derivatives, homologs or analogs thereof, can be used to detect α1β1 integrin in a variety of samples. Such samples include substances, matrices, solutions, tissues, cells, organisms, and anything else which can contain, express or be associated with α1β1 integrin. According to one embodiment, samples which carry α1β1 integrin can comprise a plurality of immobilized peptides or cells. Methods for immobilizing peptides are well known to those skilled in the art and include, for example, immobilization of peptides on nitrocellulose. Similarly, methods for immobilizing cells are well known to those skilled in the art and include, for example, immobilization of cells by cross-linking to a solid substrate. A sample can comprise a population of cells or a tissue expressing α1β1 integrin. Detection of α1β1 integrin can occur in vitro or in vivo.

The invention thus provides methods of detecting α1β1 integrin in a sample. In one embodiment, a sample is immobilized on a solid support, such as a polyacrylamide gel or nitrocellulose filter. A labeled KTS disintegrin, or a biologically active fragment, derivative, homolog or analog thereof, is then contacted with the immobilized sample for a time sufficient to allow binding of the labeled compound to any α1β1 integrin that can be present in the sample. The label can be a substance which is radioactive; emits fluorescent light; or is detectable, for example by visible, infrared or UV light; by exposure of photographic or X-ray film; by gamma camera, scintillation counter, or other device capable of detecting radioactive decay. Fluorescent (e.g., fluorescein isothiocyanate) or radioactive (e.g., ³⁵S or ¹²⁵I) labels are preferred.

Labeled KTS-disintegrins, or biologically active fragments, derivatives, homologs or analogs thereof, bound to α1β1 integrin can be detected by any appropriate means, including, for example, fluorescence microscopy, light microscopy, confocal microscopy, electron microscopy, phosphorimaging, autoradiography, scintillation counting, and nuclear magnetic resonance. Such detection techniques are well known to those skilled in the art. Various techniques for detecting the labeled compounds of the invention can also be used in conjunction with other approaches such as fluorescence activated cell sorting (FACS), flow cytometry, or endoscopic techniques.

Labeled compounds of the invention can also be used to map the interactions of α1β1 integrin with extracellular matrix proteins through techniques such as fluorescence resonance energy transfer (for example as described in Golbik et al., J. Mol. Biol. 2000, 297: 501-509, the entire disclosure of which is incorporated herein by reference).

The invention also provides a method of isolating α1β1 integrin from a sample. The α1β1 integrin can be free or bound to a cell membrane. Samples containing, or suspected of containing, α1β1 integrin can be contacted with a KTS disintegrin, or a biologically active fragment, derivative, homolog or analog thereof, that has been modified with a selectable label which allows the compound (and any bound α1β1 integrin) to be separated from the sample. The compound of the invention modified with a selectable label is then contacted with the sample for a time sufficient to allow it to bind to any α1β1 integrin present in the sample. Examples of suitable selectable labels include, for example, fluorescent labels (e.g., fluorescein isothiocyanate), magnetic particles or beads, ligands, antibodies, and polymeric or macromolecular structures (including solid supports). In preferred embodiments, the conjugated α1β1 integrin-inhibiting compound is immobilized on a solid support such as nitrocellulose, a polyacrylamide gel or a chromatography column. Once bound to the α1β1 integrin, the modified compound of the invention can be removed from the sample by any appropriate means. For example, compounds of the invention conjugated to magnetic beads, and having bound α1β1 integrin, can be removed from a sample by application of a magnetic field. Alternatively, compounds of the invention conjugated to insoluble substances, and having bound α1β1 integrin, can be removed from a sample by column chromatography, filtration, or by centrifugation. Any α1β1 integrin bound to the immobilized compound of the invention can be isolated by recovering and analyzing the complex, or by dislodging the α1β1 integrin from the immobilized compound after unbound material has been removed. Additionally, cells bound to compounds of the invention which have been modified with a fluorescent label (such as fluorescein isothiocyanate) can be isolated with flow cytometry techniques. A preferred flow cytometry technique is FACS.

Unknown peptides that bind to the compounds of the invention can be isolated and identified by techniques well known to those skilled in the art. Techniques for protein identification include, for example, SDS-PAGE separation of peptides in conjunction with silver staining or other means for detecting proteins in a gel. Techniques for detecting proteins in a gel are disclosed by Sambrook et al., Molecular Cloning: A Laboratory Manual, Cold Spring Harbor Laboratories, Second Ed., (1989), the disclosure of which is herein incorporated by reference. Unknown proteins can also be identified by sequencing, for example with automated sequencing techniques well known to those of ordinary skill in the art.

The present invention also provides antibodies against a KTS disintegrin, or a biologically active fragment, derivative, homolog or analog thereof. The antibody of the invention can, for example, specifically bind an epitope of obtustatin or viperisrastatin. The antibody can be a monoclonal antibody or a polyclonal antibody or an antibody fragment that is capable of binding antigen. In one embodiment, the invention provides a hybridoma that produces a monoclonal antibody which specifically binds to a peptide or peptide fragment according to the invention. The antibodies of the invention can also comprise antibodies, and preparations thereof, preferably produced by immunizing an animal with substantially pure obtustatin, viperisrastatin, or an immunogenic fragment thereof.

The present invention includes chimeric, single chain, and humanized antibodies, as well as Fab fragments and the products of a Fab expression library. Antibody fragments, such as Fab antibody fragments, which retain some ability to selectively bind to the antigen of the antibody from which they are derived, can be made using well known methods in the art. Such methods are generally described in U.S. Pat. No. 5,876,997 the entire disclosure of which is incorporated herein by reference. Polyclonal antibodies can be generated against the compounds of the invention. Antibodies can be obtained following the administration of one or more peptides, fragments, derivatives, or homologs to an animal, using the techniques and procedures known in the art.

Monoclonal antibodies can be prepared using the method of Mishell, B.B., et al., Selected Methods In Cellular Immunology, (W. H. Freeman, ed.) San Francisco (1980), the disclosure of which is herein incorporated by reference. Briefly, a compound of the invention is used to immunize spleen cells of Balb/C mice. The immunized spleen cells are fused with myeloma cells. Fused cells containing spleen and myeloma cell characteristics are isolated by growth in HAT medium, a medium which kills both parental cells, but allows the fused products to survive and grow. Antibodies can be used to purify the compounds of the invention, using immunoaffinity techniques within the skill in the art.

The invention will now be illustrated with the following non-limiting examples.

### EXAMPLE 1

Primary Purification of Obtustatin

Lyophilized Vipera lebetina obtusa venom was purchased from Latoxen (Valance, France). Lyophilized venom was dissolved in 0.1% trifluoroacetic acid to a final concentration of 30 mg/ml. The solution was then centrifuged for 5 minutes at 5000 rpm to remove insoluble matter. The supernatant was next applied to a C₁₈ HPLC column and the pellet was discarded. Peptides were eluted from the column with a linear 0-80% acetonitrile in H₂O gradient run which ran for 45 minutes. Peptide elution was followed by monitoring A_{206 nm} and 19 fractions were collected. Each fraction was lyophylized and resuspended in H₂O. Protein concentration in each resuspended fraction was measured using the bicinchoninic acid (BCA) assay. Five μg of protein from each fraction was then assayed for the ability to disrupt α1β1 integrin-mediated adhesion of α1K562 cells to immobilized collagen IV by the method described in Example 4. The fifth fraction contained a disintegrin activity which inhibited α1β1 integrin-mediated adhesion of α1K562 cells to immobilized collagen IV. The disintegrin activity in this fraction elutes at approximately 21 minutes and 37% acetonitrile as shown in FIG. 1. This disintegrin containing fraction was collected and lyophilized.

### EXAMPLE 2

Secondary Purification of Obtustatin

One mg of the lyophilized, disintegrin containing fifth fraction collected as described in Example 1 was dissolved in 500 μL of 0.1% trifluoroacetic acid solution and applied to a C₁₈ HPLC column. Peptides were then eluted from the column with a linear 20-70% acetonitrile in H₂O gradient run which ran for 70 minutes as shown in FIG. 2. Peptide elution was followed by monitoring A_{206 nm}. The first major peak eluted at approximately 23 minutes and contained a disintegrin activity which inhibited α1β1 integrin-mediated adhesion of α1K562 cells to immobilized collagen IV, as described in Example 4. SDS-PAGE and mass spectrometry confirmed that only one major peptide species was present in this fraction. Mass spectrometry revealed the eluted obtustatin peptide had a molecular mass of 4395 Da. The primary amino acid sequence of obtustatin was determined by automated Edman degradation, and is given in SEQ ID NO: 1. The yield after the primary and secondary purifications was approximately 12 mg substantially purified obtustatin per 1 g crude Vipera lebetina obtusa venom.

### EXAMPLE 3

Purification of Viperisrastatin

Viperisrastatin was isolated from lyophilized Vipera palestinae venom using the same conditions, reagents and techniques described above for isolation of obtustatin. The primary amino acid sequence of viperisrastatin was determined by automated Edman degradation, and is given in SEQ ID NO: 2.

### EXAMPLE 4

The Effect of Obtustatin, EP-obtustatin and Fragments of Obtustatin on the Adhesion of α1K562 Cells to Collagen IV

α1K562 cells express the α1β1 integrin which binds collagen IV. Ethylpyridylated obtustatin (EP-obtustatin) is a form of full-length obtustatin in which the S-S bonds between cysteine residues have been reduced and exposed thiol groups have been alkylated by reaction with 4-vinylpyridine.

EP-obtustatin was generated by incubating an EP-obtustatin reaction cocktail for 12 hours in the dark at room temperature. The EP-obtustatin reaction cocktail consisted of 100 μg of obtustatin in a solution of 6 M guanidine.HCl, 4 mM EDTA, 0.1 M Tris.HCl (pH 8.5), 3.2 mM dithiothreitol (DTT), and 1 μL of a 95% pure 4-vinylpyridine solution added per 100 μL of each reaction cocktail to start the reaction. Purified EP-obtustatin was obtained post-reaction by applying the reaction cocktail to a C₁₈ HPLC column followed by elution from the column with a linear 0-80% acetonitrile in 0.1% (v/v) trifluoroacetic acid/H₂O gradient which ran for 45 minutes. Peptide elution was followed by monitoring A_{206 nm} and the EP-obtustatin containing fraction was collected, lyophilized and resuspended in H₂O.

Peptides with the primary amino acid sequences of CWKTSLTSHYC (SEQ ID NO: 5), TSLTS (SEQ ID NO: 6), and CKLKPAGTTC (SEQ ID NO: 7) were generated with standard synthetic techniques. These peptides were based on the primary sequence of full-length obtustatin.

The ability of EP-obtustatin and the synthetic peptides described above to inhibit activity of the α1β1 integrin was then assayed. Collagen IV (0.2 μg/well) in 0.02 M acetic acid was immobilized by incubation overnight at 4° C. on a 96-well plate. Plates were then blocked with 1% (w/v) BSA (bovine serum albumin) in Hank's Balanced Salt Solution (HBSS) containing 3 mM Mg²⁺ at room temperature for 1-2 hours to prevent cells from non-specifically binding to the plates. α1K562 cells were labeled by incubation with 12.5 μM 5-chloromethylfluorescein diacetete (CMFDA) in HBSS for 15 minutes at 37° C. CMFDA labeled α1K562 cells were then pelleted, washed and resuspended in HBSS buffer containing 3 mM Mg²⁺ and 1% BSA. 1×10⁵ CMFDA labeled cells were then added to each well in the presence or absence of full-length obtustatin, EP-obtustatin, or the synthetic peptides and incubated at 37° C. for 30 minutes. The carrier for full-length obtustatin, EP-obtustatin, and the synthetic peptides was H₂O containing 1% (w/v) BSA. The plates were washed three times with HBSS containing 3 mM Mg²⁺ and 1% (w/v) BSA to remove unbound cells. Bound cells were lysed using 0.5% (v/v) Triton X-100 in H₂O. Fluorescent CMFDA released by lysis of adherent cells in a given well was read using a Cytofluor 2350 fluorescence plate reader.

Under these assay conditions full-length, native obtustatin had an IC₅₀ value of 2 nM (FIG. 4) and full-length, reduced EP-obtustatin had an IC₅₀ value of 30 μM. The synthetic peptides limited α1β1 integrin-mediated adhesion of α1K562 cells to collagen IV to lesser extent (see FIG. 4): The peptide of sequence CWKTSLTSHYC (SEQ ID NO: 5) had an IC₅₀ value of 600 μM; the peptide of sequence TSLTS (SEQ ID NO: 6) had an IC₅₀ value of 3.5 mM; and the peptide of sequence CKLKPAGTTC (SEQ ID NO: 7) did not appreciably inhibit α1β1 integrin-mediated adhesion of α1K562 cells to collagen IV.

### EXAMPLE 5

The Effect of Obtustatin on Adhesion of MV3 Cells to Immobilized Collagen IV

The α1β1 integrin specific inhibitory effect of obtustatin was confirmed using human melanoma MV3 cells. MV3 cells express both the α1β1 and α2β1 integrins. MV3 cells adhere strongly to collagen IV through α1β1 and α2β1 integrin-mediated interactions.

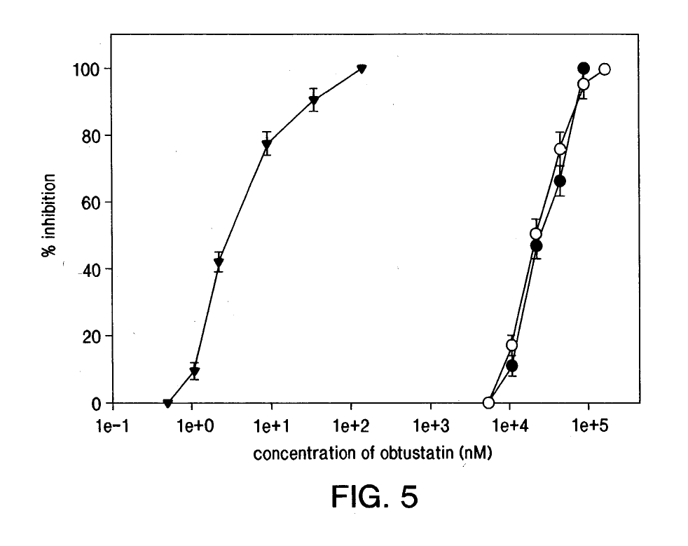

Collagen IV (0.2 μg/well) in 0.02 M acetic acid was immobilized overnight at 4° C. on a 96-well plate. Plates were then blocked with 1% (w/v) BSA (bovine serum albumin) in Hank's Balanced Salt Solution (HBSS) containing 3 mM Mg²⁺ at room temperature for 1-2 hours to prevent cells from non-specifically binding to the plates. α1K562 cells were labeled by incubation with 12.5 μM 5-chloromethylfluorescein diacetete (CMFDA) in HBSS for 15 minutes at 37° C. CMFDA labeled MV3 cells were then pelleted, washed and resuspended in HBSS buffer containing 3mM Mg²⁺ and 1% (w/v) BSA. 1×10⁵ cells were then added to each well in the presence (10 μg/ml) or absence of the α1 integrin subunit specific AJH10 mAb and α2 integrin subunit specific P1E6 mAb as indicated in FIG. 5. Obtustatin was added at the concentrations indicated in FIG. 5 simultaneously with the mAbs. The plates were then incubated at 37° C. for 30 minutes and washed three times with HBSS containing 3 mM Mg²⁺ and 1% (w/v) BSA to remove unbound cells. Bound cells were lysed using 0.5% (v/v) Triton X-100 in H₂O. Fluorescent CMFDA released by lysis of adherent cells in a given well was read using a Cytofluor 2350 fluorescence plate reader.

As indicated in FIG. 5, obtustatin specifically inhibited α1β1 integrin-mediated adhesion of MV3 cells to immobilized collagen IV. Adhesion of P1E6 blocked MV3 cells, in which only the α1β1 integrin mediates adhesion to collagen IV, was inhibited by significantly lower obtustatin concentrations than AJH10 blocked cells or unblocked cells. In contrast, unblocked MV3 cells and AJH10 blocked cells, which can adhere to collagen IV via the α2β1 integrin, were comparatively insensitive to obtustatin. The extreme obtustatin sensitivity of P1E6 blocked MV3 cells, in combination with the relative insensitivity of unblocked and AJH10 blocked MV3 cells to this peptide, reveals that obtustatin is a potent and selective inhibitor of α1β1 integrin.

Control experiments confirmed that only the α1β1 and α2β1 integrins mediate adhesion of MV3 cells to collagen IV. These control experiments revealed that at final concentrations of 200 μg/ml neither the P1E6 antibody or AJH10 antibody alone could inhibit α1β1 and α2β1 integrin-mediated adhesion of MV3 cells to collagen IV. Yet simultaneous incubation of MV3 cells with P1E6 and AJH10 antibodies at final concentrations of 10 μg/ml each entirely inhibited α1β1 and α2β1 integrin-mediated adhesion of MV3 cells to collagen IV. Additionally, mAb ASC-1 directed against the α3 integrin subunit present in the VLA-3 collagen receptor had no effect, either alone or in combination with the P1E6 and AJH10 mAbs, on the binding of MV3 cells to collagen IV. Together the results of these control experiments indicate that only the α1β1 and α2β1 integrins mediate adhesion of MV3 cells to collagen IV.

### EXAMPLE 6

Comparison of the Effects of Obtustatin Echistatin, and Eristostatin on the Binding of Selected Integrins

Adhesive ligands for the selected integrins consisted of collagen IV (0.2 μg/well), collagen I, fibronectin, or vascular cell adhesion molecule-1 (VCAM-1). Adhesive ligands in 0.02 M acetic acid were immobilized overnight at 4° C. on a 96-well plate. Plates were then blocked with 1% (w/v) BSA (bovine serum albumin) in Hank's Balanced Salt Solution (HBSS) containing 3 mM Mg²⁺ at room temperature for 1-2 hours to prevent cells from non-specifically binding to the plates. α1K562, α2K562, K562, A5, and Jurkat cells expressing the α1β1, α2β1, α5β1, αIIbβ3, and α4β1 integrins respectively were labeled by incubation with 12.5 μM 5-chloromethylfluorescein diacetate (CMFDA) in HBSS for 15 minutes at 37° C. CMFDA labeled cells were then pelleted, washed and resuspended in HBSS buffer containing 3 mM Mg²⁺ and 1% (w/v) BSA. 1×10⁵ cells were then added to the various wells and the amount of either obtustatin, echistatin, or eristostatin was gradually increased up to determine the IC₅₀ values indicated below in Table 5. Plates were then incubated at 37° C. for 30 minutes as indicated in Table 5 and washed three times with HBSS containing 3 mM Mg²⁺ and 1% (w/v) BSA to remove unbound cells. Bound cells were lysed using 0.5% (v/v) Triton X-100 in H₂O. Fluorescent CMFDA released by lysis of adherent cells in a given well was read using a Cytofluor 2350 fluorescence plate reader.

The IC₅₀ values in Table 5 represent the mean of three independent experiments. The results summarized in Table 5 reveal that obtustatin is a potent and selective inhibitor of α1β1 mediated binding to collagen IV.

TABLE 5
Comparison of the effects of obtustatin, echistatin, and
eristostatin on the binding of selected integrins.

| Cell | | | | | |
|---|---|---|---|---|---|
| Suspen- | | Adhesive | IC₅₀, nM | | |
| sion | Integrin | Ligand | Obtustatin | Echistatin | Eristostatin |
| α1K562 | α1β1 | Collagen IV | 2 | >10,000 | >10,000 |
| α2K562 | α2β1 | Collagen I | >10,000 | >10,000 | >10,000 |
| K562 | α5β1 | Fibronectin | >10,000 | 50 | >10,000 |
| A5 | αIIbβ3 | Fibrinogen | >10,000 | 50 | 5 |
| Jurkat | α4β1 | VCAM-1 | >10,000 | >10,000 | >10,000 |

### EXAMPLE 7

Effect of Obtustatin and Eristostatin on Angiogenesis in Japanese Quail CAM Assay

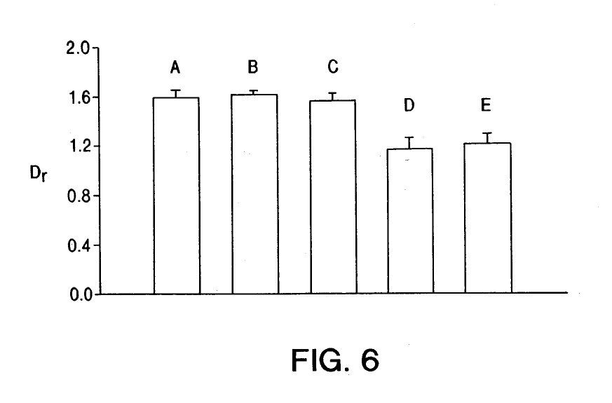

The disintegrins obtustatin and eristostatin were evaluated for their effect on angiogenesis via a Japanese quail chorioallantoic membrane (CAM) assay, as is known in the art (see, for example, Parsons-Wingerter P et al., “A Novel Assay of Angiogenesis in the Quail Chorioallantoic Membrane: Stimulation by bFGF and Inhibition by Angiostatin According to Fractal Dimension and Grid Intersection.” Microvascular Research 55(3):201-214, 1998, the disclosure of which is herein incorporated by reference). The extent of angiogenesis was assessed by evaluating the pattern of vessel branching in skeletonized quasi-two-dimensional CAM vasculature, represented as the fractal dimension Dᵣ. FIG. 6 shows the results for phosphate buffered saline (control; bar A); 20 μg eristostatin (bar B); 50 μg eristostatin (bar C); 20 μg obtustatin (bar D); and 50 μg obtustatin (bar E).

These results show that obtustatin at 20 μg and 50 μg exhibits potent inhibitory activity (up to 30% of control) on angiogenesis. The assay was performed without any angiogenesis stimulators, such as growth factors, indicating the potency of obtustatin in inhibiting angiogenesis. Eristostatin at either 20 μg and 50 μg showed no affect on angiogenesis in vivo.

### EXAMPLE 8

Effect of Single Mutation within Synthetic Peptides Representing the Integrin-binding Loop of Obtustatin on Adhesion of α1K562 Cells to Collagen IV.

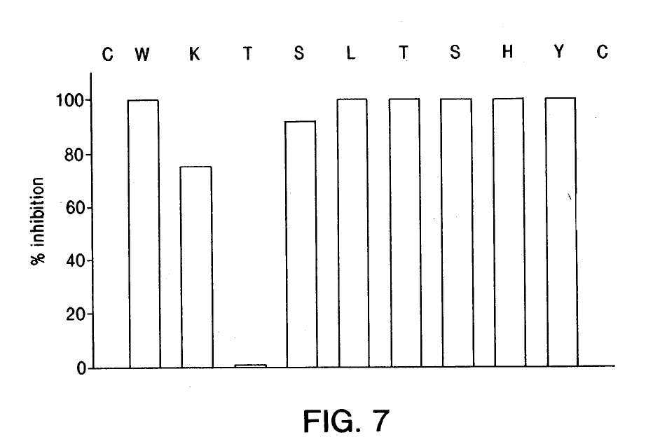

As discussed above, the motif in obtustatin analogous the RGD sequence in other disintegrins could be theoretically identified as SL, SLT, TSL or KTS. To identify the amino acids involved in the activity of obtustatin, nine peptides were synthesized commercially (Sigma-Genosis) based on CWKTSLTSHYC (SEQ ID NO:5), which is the native obtustatin sequence containing the integrin binding loop. Each synthetic peptide contained a single change of one of the native amino acids to alanine, as follows. The C residues on either end of the native peptide were not converted. Peptide 1 had the W converted to alanine with all other amino acids as in the native sequence; peptide 2 had the K adjacent to the W converted to alanine, with all other residues as in the native sequences, etc. The collagen IV binding activity of each synthetic peptide, at a final assay concentration of 1 mM, was measured as described in Example 5 above. The results for each peptide are shown in FIG. 7 under, the appropriate converted amino acid, given as % inhibition of adhesion of α1K562 cells to collagen IV.

The results show that the K, T and S adjacent to the W are involved in obtustatin binding to collagen IV, and that the T is the most important of the three with respect to the binding activity of obtustatin. These results suggest that the KTS sequence can be relevant for binding of obtustatin to α1β1 integrin.

### EXAMPLE 9

Effect of Obtustatin and Echistatin on Lewis Lung Carcinoma Growth in Syngeneic Mouse Model.

Lewis lung carcinoma cells (1×10⁶) were injected under the skin of the C57BL/6 mice (Tacoma Inc., Germantown, N.Y.), and tumors were allowed to grow for one week. The average tumor size after one week of growth was approximately 0.08 cm³. The one-week time-point was called “day 0.” Two treatment groups of four mice each were injected intraperitoneally once daily with either 5 mg/kg obtustatin or 5 mg/kg echistatin beginning on day 0 for a total of eight days. A control group of four mice received only PBS. Tumor volume was measured daily using the formula length×width²×0.52.

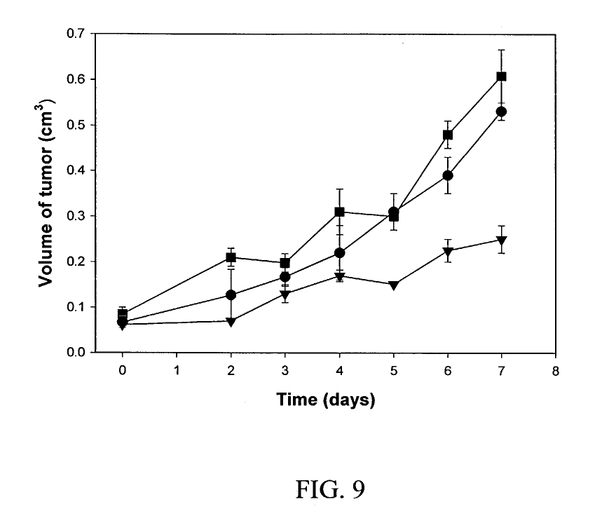

As shown in FIG. 9, obtustatin inhibited tumor development in this model by about half, whereas no effect of echistatin was observed. Echistatin is a RGD-containing disintegrin, and has been recognized as a potent inhibitor of α3β1 and α5β1 integrin. These data suggest that α1β1 integrin might play a more significant role in cancer progression than RGD-dependent integrins (such as α3β1 and α5β1 integrins) that have previously been implicated in angiogenesis and cancer development.

### EXAMPLE 10

Effect of Obtustatin on Apoptosis of Dermal Microvascular Endothelial Cells and Human Melanoma Cells

The mechanisms of the cancer-suppressing effect observed for obtustatin were investigated as follows. Dermal microvascular endothelial cells (EC) and human melanoma cells (MV3) were cultured and exposed to TNF-α, vincristine and obtustatin. (TNF-α and vincristine are compounds which induce cell death by apoptosis in both normal and cancerous cells.) Control cells received only vehicle. The extent of apoptosis was measured by determining caspase-3 levels.

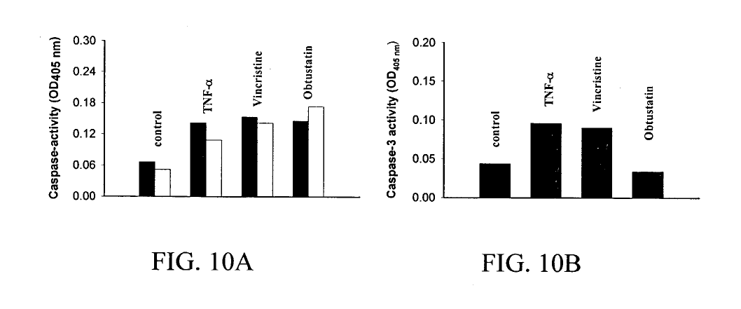

As shown in FIGS. 10A-10B, TNF-α and vincristine induced apoptosis in both EC and MV3 cells. However, obtustatin induced significant apoptosis in EC but not in MV3 cells. These data suggest that obtustatin exerts an anticancer effect by inhibiting proliferation of normal endothelial cells, which in turn blocks angiogenesis and solid tumor progression.

### EXAMPLE 11

Effect of Obtustatin on Growth of Human Microvascular Endothelial Cells and Human Melanoma Cells

Human microvascular endothelial cells (HMVECs) and melanoma cells (HS.939T) were cultured and treated with vehicle (control), 2 μM obtustatin, 2 μM echistatin or 50 ng/ml TNF-α. Growth of the cells was determined by a standard BrdU incorporation assay (Chemicon), according to the manufacturer's instructions.

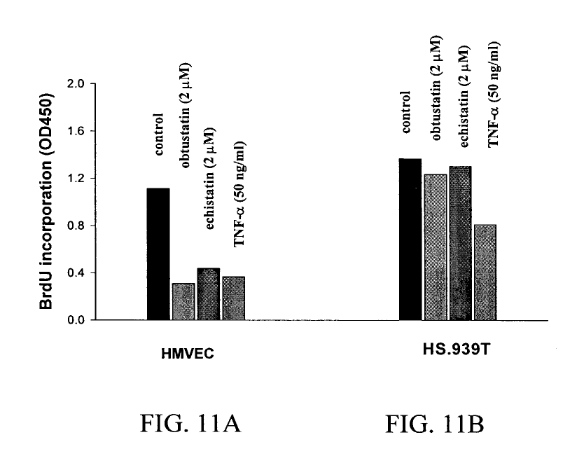

As shown in FIGS. 11A and 11B, obtustatin inhibited growth of HMVECs, but not the HS.939T melanoma cells. Taken together with the results of Example 9, these data indicate that obtustatin exerts an anticancer effect by inhibiting proliferation of normal endothelial cells to block angiogenesis, and not by direct inhibition of cancer cell growth.

### EXAMPLE 12

Effect of Obtustatin on Human Microvascular Endothelial Cell Tube Formation in Matrigel™

An endothelial cell tube formation assay was performed to assess the anti-angiogenic effect of obtustatin in vitro as follows. Twenty-four well plates were coated with a 1:2 dilution of Matrigel™ in EBM-2 medium, which was polymerized by incubation for 1 hour at 37° C. HMVECs (1.2×10⁵ cells per well) were added onto the polymerized Matrigel™ (BD Biosciences, Lexington, Ky.) in EBM-2 medium, in the absence or presence of 60 μM obtustatin. The plates were incubated for 24 hours at 37° C., and analyzed for the extent of tube formation by observation with an inverted microscope, using a magnification of 10× ocular and 10× objective. The formation of HMVEC tubes on Matrigel™ was completely abolished by the presence of obtustatin at a concentration of 60 μM.

### EXAMPLE 13

Effect of Obtustatin on Melanoma Cell Migration into Collagen IV or FGF Through HUVEC Layer

Cell migration through an endothelial cell layer is an important event in cancer cell metastasis. The effect of obtustatin on melanoma cell migration through an endothelial cell layer in vitro was evaluated as follows. Human umbilical vein endothelial cells (HUVECs) of 3-7 passages were purchased from Cascade Biologics (Portland, Oreg.). The HUVECs were seeded into 8.0 μm pore size FALCON® HTS FluoroBlok™ inserts (Becton Dickinson, Franklin Lakes, N.J.) in serum-containing endothelial cell growth medium. The inserts were placed into the wells of a 24-well plate (Becton-Dickinson), and the HUVECs allowed to grow to confluence on the insert over 72 hours. The interior of the FluoroBlok™ inserts is referred to below as the “upper chamber,” and the interior of the well in which the insert sits is referred to as the “lower chamber.”

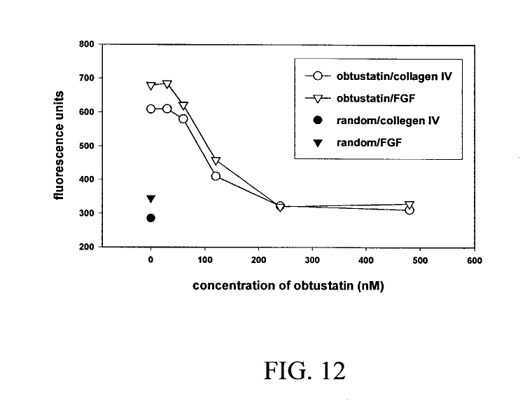

Twenty-four hours before the assay, the upper chambers were washed with serum-free medium, and new medium containing TNF-α (3 ng/ml) was added to activate the HUVECs. Immediately prior the addition of MV3 human melanoma cells to the upper chambers, the upper chambers were washed with serum-free DMEM and the medium in the lower chambers was replaced with 500 μL of serum-free DMEM with or without either collagen IV or fibroblast growth factor (FGF) as a chemoattractant. MV3 cells (2×10⁵), previously labeled with the fluorescent dye 1,1′-dioctadecyl-3,3,3′,3′-tetramethyllindocarbocyanine perchlorate (“Dil”; Molecular Probes, Eugene, Oreg.) were then added to each upper chamber along with varying concentrations of obtustatin from 0 to 500 nM. A random peptide was added to certain upper chambers as a control. After 3 hours incubation at 37° C. in 5% CO₂, the extent of MV3 migration into the bottom chambers was assessed using a bottom-reading Cytofluor 2350 (Millipore Inc., Bedford Mass.). The amount of fluorescence in the lower chambers is directly related to the number of MV3 cells which have migrated through the HUVEC layer from the upper chamber. As shown in FIG. 12, obtustatin inhibited migration of MV3 cells through the HUVEC layer when collagen VI or FGF were used as chemoattractants. Essentially complete inhibition of MV3 cell migration occurred at an obtustatin concentration above 200 nM.

### EXAMPLE 14

Comparison of the Effects of Obtustatin, Viperisrastatin, Echistatin, and Eristostatin on the Binding of Selected Integrins

The effect of obtustatin, viperisrastatin, echistatin, and eristostatin on the binding of selected integrins to their adhesive ligands was evaluated using the cell adhesion assay described in Example 6. The integrin-expressing cells, integrins and corresponding adhesive ligands, and the results of the cell adhesion assay are shown in Table 6.

TABLE 6
Comparison of inhibitory effects of short disintegrins
on the binding of selected integrins

| Cell | | | IC₅₀ (nM) | | | |
|---|---|---|---|---|---|---|
| Suspension | Integrin | Ligand | Obtustatin | Viperisrastatin | Echistatin | Eristostatin |
| α1K562 | α1β1 | Coll IV | 2 | 0.08 | >10,000 | >10,000 |
| α1K562 | α1β1 | Coll I | 0.5 | 0.02 | >10,000 | >10,000 |
| HS.939T | α1β1 | Coll IV | 0.1 | 0.002 | >10,000 | >10,000 |
| B16F10 | α1β1 | Coll IV | 0.2 | 0.25 | >10,000 | >10,000 |
| α2K562 | α2β1 | Coll I | >10,000 | >10,000 | >10,000 | >10,000 |
| α6K562 | α6β1 | LM | >10,000 | >10,000 | >10,000 | >10,000 |
| K562 | α5β1 | FN | >5,000 | >10,000 | 50 | >10,000 |
| Jurkat | α4β1 | VCAM-1 | >10,000 | >10,000 | >10,000 | >10,000 |
| SW480α9 | α9β1 | VCAM-1 | >10,000 | >10,000 | >10,000 | >10,000 |
| A5 | αIIbβ3 | FG | >10,000 | >10,000 | 50 | 50 |
| JY | αvβ3 | VN | >10,000 | >10,000 | 0.1 | 2,000 |

The data represent the mean of the results of three experiments. Coll = collagen, LM = laminin, FN = fibronectin, VCAM-1 = vascular cell adhesion molecule-1, FG = fibrinogen, VN = vitronectin.

As can be seen from Table 6, obtustatin and viperisrastatin inhibit binding of cells expressing α1β1 integrin to collagen I or IV, whereas echistatin and eristostatin do not. The activity of viperisrastatin is from 1-2 orders of magnitude greater than obtustatin with respect to human α1β1 integrin (see data for binding of α1K562 cells HS.939T cancer cells, both of which express human α1β1 integrin. However, the activity of obtustatin and viperisrastatin was similar with respect to binding of mouse α1β1 integrin (see data for B16F10 cells, which express mouse α1β1 integrin).

All references discussed herein are incorporated by reference. One skilled in the art will readily appreciate that the present invention is well adapted to carry out the objects and obtain the ends and advantages mentioned, as well as those inherent therein. The present invention can be embodied in other specific forms without departing from the spirit or essential attributes thereof and, accordingly, references should be made to the appended claims, rather than to the foregoing specification, as indicating the scope of the invention.

What is claimed is:

1. A compound comprising SEQ ID NO: 1, or a biologically active fragment, derivative, homolog, analog of SEQ ID NO: 1, or a pharmaceutically acceptable salt of any of them.

2. The compound of claim 1 comprising SEQ ID NO: 1.

3. The compound of claim 1 comprising SEQ ID NO: 2.

4. A compound according to claim 1, which comprises a core sequence of Lys-Thr-Ser, has greater than about 80% sequence identity with SEQ ID NO: 1, and inhibits α1β1 integrin-mediated cellular adhesion to collagen IV.

5. The compound of claim 4, which has greater than about 90% sequence identity with SEQ ID NO: 1.

6. The compound of claim 1, comprising a peptide fragment of five to thirty amino acids in length.

7. A compound according to claim 1, comprising the sequence X₁-Lys-Thr-Ser-X₂, wherein
X₁ is from zero to twenty-five amino acids; and
X₂ is from zero to twenty amino acids, and wherein the compound inhibits α1β1 integrin-mediated cellular adhesion to collagen IV.

8. The compound of claim 7, wherein,
X₁ is
(i) zero amino acids, or
(ii) the segment Cys-Thr-Thr-Gly-Pro-Cys-Cys-Arg-Gln-Cys-Lys-Leu-Lys-Pro-Ala-Gly-Thr-Thr-Cys-Trp, or an amino-terminal truncation fragment thereof containing at least one amino acid, and
X₂is
(i) zero amino acids, or
(ii) the segment X₄-Thr-Ser-His-Tyr-Cys-Thr-Gly-Lys-Ser-Cys-Asp-Cys-Pro-X₅-Tyr-X₆-Gly or a carboxy-terminal truncation fragment thereof containing at least one amino acid, wherein X₄ is Leu or Arg, X₅ is Leu or Val, and X₆ is Pro or Gln;
and provided that when X₄ is Leu then X₅ is Leu and X₆ is Pro, and when X₄ is Arg then X₅ is Val and X₆ is Gln.

9. The compound of claim 8, wherein X₄ is Leu.

10. The compound of claim 8, wherein X₄ is Arg.

11. A compound comprising the sequence Cys-Xaa-Xaa-Xaa-Xaa-Cys-Cys-Xaa-Xaa-Cys-Xaa-Xaa-Xaa-Xaa-Xaa-Xaa-Xaa-Xaa-Cys-Xaa-Lys-Thr—Ser-Xaa-Xaa-Xaa-Xaa-Xaa-Cys-Xaa-Xaa-Xaa-Xaa-Cys-Xaa-Cys-X₇, or a pharmaceutically acceptable salt thereof, wherein X₇ is zero or any 1, 2, 3, 4, or 5 amino acids, and each Xaa is independently any amino acid, and wherein the compound inhibits α1β1 integrin-mediated cellular adhesion to collagen IV.

12. A composition comprising the compound of claim 1 and a pharmaceutically acceptable carrier.

13. A composition comprising the compound of claim 7 and a pharmaceutically acceptable carrier.

14. A composition comprising the compound of claim 11 and a pharmaceutically acceptable carrier.

15. An antibody which specifically binds to SEQ ID NO: 1.

16. The antibody of claim 15 which is a monoclonal antibody.

17. An antibody which specifically binds to SEQ ID NO: 2.

18. The antibody of claim 17 which is a monoclonal antibody.

19. A method of inhibiting the binding of α1β1 integrin to its adhesive ligand, comprising contacting a sample with an effective amount of a compound according to claim 1 so that the binding of α1β1 integrin to its adhesive ligand is inhibited.

20. The method of claim 19, wherein the compound is obtustatin or viperisrastatin.

21. The method of claim 19 wherein the sample comprises cells α1β1 integrin not bound to a cell membrane.

22. The method of claim 19, wherein the sample comprises cells expressing α1β1 integrin.

23. A method of treating a disease or biological condition associated with the binding of α1β1 integrin to its adhesive ligand, comprising administering to a subject an amount of a compound according to claim 1 sufficient to inhibit the binding of α1β1 integrin with their ligands.

24. The method of claim 23, wherein the compound is obtustatin or viperisrastatin.

25. The method of claim 23 wherein the disease is selected from the group consisting of insulin dependent diabetes mellitus, multiple sclerosis, rheumatoid arthritis, ulcerative colitis, arteriosclerosis, and cancer.

26. The method of claim 23 wherein the biological condition is selected from the group consisting of thrombic occlusion formation, blood clot formation, wound healing, allergy, organ rejection, asthma, neovascularization, restenosis of arteries, and angiogenesis.

27. The method of claim 26 wherein said biological condition is angiogenesis.

28. The method of claim 27 wherein the angiogenesis is associated with metastasis, comeal graft rejection, ocular neovascularization, retinal neovascularization, diabetic retinopathy, retrolental fibroplasia, neovascular glaucoma, gastric ulcer, infantile hemangiomas, angiofibroma of the nasopharynx, avascular necrosis of bone, or endometriosis.

29. A method of detecting α1β1 integrin in a sample, comprising:
(a) contacting the sample with a compound according to claim 1 which is modified with a label, for a time sufficient to allow binding of the labeled compound to any α1β1 integrin present in the sample; and
(b) detecting the labeled compound bound to the α1β1 integrin.

30. The method of claim 29, wherein the compound is obtustatin or viperisrastatin.

31. The method of claim 29 wherein the label is selected from the group consisting of radioactive compounds, compounds that emit fluorescent light, compounds detectable by visible light, compounds detectable by infrared light, compounds detectable by UV light; compounds detectable by exposure of photographic film, compounds detectable by exposure of X-ray film, compounds detectable by gamma camera, and compounds detectable by scintillation counter.

32. The method of claim 29 wherein the sample is immobilized on a solid support.

33. The method of claim 29 wherein the sample comprises a plurality of unknown peptides.

34. The method of claim 28 wherein the sample comprises cells.

35. A method of isolating α1β1 integrin from a sample, comprising:
(a) contacting the sample with a compound according to claim 1 which is modified with a selectable label, for a time sufficient for the selectable label-modified compound to bind to any α1β1 integrin present in the sample; and
(b) separating the selectable label-modified compound bound to α1β1 integrin from the sample.

36. The method of claim 35, wherein the KTS-disintegrin is obtustatin or viperisrastatin.

37. The method of claim 35, wherein said sample comprises a plurality of unknown peptides.

38. The method of claim 35, wherein said sample comprises cells.

39. The method of claim 38 wherein the selectable label used to modify the KTS-disintegrin is fluorescein isothiocyanate, and the α1β1 integrin expressing cells are isolated by flow cytometry.
